(19) United States
(12) Patent Application Publication
(10) Pub. No.: US 2005/0053596 A1
(43) Pub. Date: Mar. 10, 2005

(54) Therapeutic biological product and method for formation of new vascularised bone

(76) Inventor: Bradley Michael John Stringer, Cynocoed , Cardiff (GB)

Correspondence Address: KLAUBER & JACKSON, 411 HACKENSACK AVENUE, HACKENSACK, NJ, 07601, US

(21) Appl. No.: 10/492,058

(22) PCT Filed: Oct. 9, 2002
(86) PCT No.: PCT/GB02/04576

(30) Foreign Application Priority Data
Oct. 9, 2001 (US) 09973484

(51) Int. Cl. A61K 38/48

(52) U.S. Cl. 424/94.63

(57) ABSTRACT

An extracellular material in freeze-dried form obtained from skeletal cells which has osseoinductive bone repair and regeneration activity in vivo, and compositions and methods involving the material.

### BACKGROUND OF THE INVENTION

Replacement of lost bone is the challenge facing orthopaedic surgeons, neurosurgeons, craniofacial surgeons, and periodontists all over the world today.

The surgical repair of lost bone is a problem faced by dentists with patients suffering from periodontal disease, for example. Periodontal disease is one of the most prevalent afflictions, one consequence of which is alveolar bone loss, which in itself is a major disease entity. Presently periodontists and patients work together in treating the symptoms of periodontal disease, and effective techniques that predictably promote the body's natural ability to regenerate lost periodontal tissues (particularly alveolar bone) still need to be developed.

In another field of dentistry, Dental Implantology, a great deal of biomaterial research is being conducted in an attempt to determine factors or substances that can improve the quality of bone to implant contact (osteointegration).

In recent decades a surge in research into understanding bone formation and bone healing has led to the development of various techniques to promote bone healing, or to replace lost bone.

Endochondral Ossification

Endochondral ossification represents the deposition of a bone matrix upon a pre-existing cartilage template and accounts for much of the skeletal formation during embryogenesis and postnatal growth. During the initial phases of this process a region comprising resting or germinal chondrocytes differentiates into a zone of proliferating chondrocytes that then hypertrophies. These hypertrophic chondrocytes become progressively larger, display more mitoses, and are more metabolically active. It is the hypertrophic chondrocytes that lay down the unmineralised and avascular cartilage matrix that is the model for developing bone.

As cell hypertrophy progresses, the pre-existing non-calcifiable and avascular cartilage matrix is transformed to a calcifiable one that is penetrable by blood vessels through angiogenesis. The invading vasculate imports mesenchymal stem cells (MSC's), haemapoietic precursors and osteoclasts. As the osteoclasts degrade the hypertrophic cartilage matrix, mesenchymal stem cells differentiate into primitive marrow cells and osteoblasts; the osteoblasts line the hypertrophic cartilage lacunae in this primary centre of ossification and deposit a bone matrix.

Endochondral ossification is a developmentally regulated process which occurs in a highly co-ordinated temporal and spatial manner, in which there is a sequential recruitment and differentiation of cells which form cartilage, vascular and bone tissues. Such sequence of events relies on the precise coupling of chondrogenesis (cartilage production) with osteogenesis (bone formation).

Hypertrophic chondrocytes play a central role in endochondral bone formation. Chondrocyte hypertrophy is intimately linked to angiogenesis, and when hypertrophy is inhibited, e.g. by parathyroid hormone-related peptide, angiogenesis and subsequent endochondral ossification is blocked, illustrating the major role of hypertrophic chondrocytes in endochondral ossification.

The process of chondrocyte maturation, in conjunction with the establishment of secondary centres of ossification at the outer (epiphyseal) ends of endochondral bone, defines the formation of a growth plate. Growth plates provide bones with longitudinal growth potential until maturity. However, endochondral ossification can be re-initiated during bone healing (e g. fracture repair).

Bone Healing

Bone heals in a unique way compared with other connective tissues. Rather than develop scar tissue, it has the ability to regenerate itself completely. Intact bone is constantly being resorbed and remodelled—a delicate balance coordinated by a rather complex cascade of cellular events.

The majority of fractures heal by secondary fracture healing and that involves a combination of intramembranous and endochondral ossification. The fracture healing sequence involves five discrete stages of healing. This includes an initial stage in which a haematoma is formed and inflammation occurs; a subsequent stage in which cartilage begins to form and angiogenesis develops, and then three successive stages of cartilage calcification, cartilage resorption and bone deposition, and ultimately a more chronic stage of bone remodelling.

It is thought that one of the functions of the haematoma is to be a source of signalling molecules which, in conjuction with others, have the capacity to initiate the cascades of cellular events that are critical to fracture healing.

Perhaps the most important response in fracture healing is that of the periosteum. Here, committed osteoprogenitor cells and uncommitted, undifferentiated mesenchymal stem cells contribute to the process of fracture healing by a recapitulation of embryonic intramembranous ossification and endochondral ossification. It is the ability of factors to stimulate differentiation of osteogenic mesenchymal stem cells, that determines the osteoinductive potential of bone graft substances.

The bone that forms by intramembranous ossification is found further from the site of the fracture, results in the formation of a hard callus, and forms bone directly without first forming cartilage. Two weeks after fracture, cell proliferation declines and hypertrophic chondrocytes become the dominant cell type in the chondroid callus. The resulting endochondral bone is formed adjacent to the fracture site.

Many of the cellular processes that occur during fracture healing parallel those that occur in the growth plate during development, except in fracture healing these processes occur on a temporal rather than a spatial scale.

Fracture healing and bone formation involve a series of distinct cellular responses that are under the control of specific paracrine and autocrine intracellular signalling pathways, it can be viewed as a well orchestrated series of biological events.

Bone Growth Factors

Bone is known to be a major source of growth factors. These growth factors have significant effects on bone and cartilage metabolism. This suggests an important role for these growth factors in mediating hormonal responses locally, and a local metabolic regulation of bone metabolism without, necessarily the influence of systemic hormones. They act in a paracrine and autocrine manneor, and are responsible for the highly coordinated manner in which bone forms remodels and regenerates after a defect occurs.

Bone derived growth factors stimulate cell replication and contribute to the stimulation of differentiation and metabolic functions of bone cells. They exhibit their effects through binding membrane bound receptors. This leads to a cascade of intracellular events that affect the expression of genes that encode for such metabolic functions as cell division and protein synthesis.

Studies have additionally revealed that cartilage also contains these growth factors suggesting that bone and cartilage interact with each other exchanging growth signals in a paracrine fashion. Hypertrophic chondrocytes also produce latent growth factors and growth factor binding proteins, which help store growth factors in their ECM. Once cleaved/activated they are able to exert an effect on target cells including hypertrophic chondrocytes (autocrine pathway).

Transforming Growth Factors (TGF-bs)

The concentration of TGF-b is 100 times higher in bone than in other tissues and osteoblasts have a higher concentration of TGF-b receptors. Along with BMP's, TGF-b belongs to the TGF-b super-family. A total of 5 subtypes of TGF-b are known (1-5). TGF-bs are multifunctional growth factors with a broad range of activities. TGF-b increases bone formation in vivo, and induces multiple cellular effects on osteoblast activity in vitro). It can either stimulate or is inhibit cell proliferation and activity depending on the cell maturation stage. For example TGF-b₁ and TGF-b₂ induce chondrogenesis and production of type II collagen (marker for articular chondrocytes) in embryonic cells, but inhibit type II collagen production in hypertrophic chondrocytes and cause them to lose their cartilaginous phenotype.

TGF-bs are important regulators of the synthesis and deposition of ECM components, and although they affect human bone marrow stromal cells, they do not alone induce complete osteogenesis in vitro. Instead they act synergistically with other growth factors. For example in vitro studies show TGF-b and PDGF stimulate osteoblast migration and TGF-b₂ and BMP-2 act in a sequential manner at different stages to promote human bone marrow stromal cell differentiation towards the osteoblast phenotype.

In bone TGF-bs are produced by osteocytes, osteoblasts, osteoclasts and chondrocytes. TGF-bs are secreted as biologically inactive precursor proteins called Latent TGF-bs. They are activated by an extremely acidic pH or by a protease (plasminogen). Osteoclasts can also activate latent TGF-b, stimulating new osteoblastic bone formation, illustrating how these two cell types with biologically opposed functions stimulate one another to regulate bone remodelling and new bone formation.

Some studies have reported contradictory findings concerning the role of TGF-bs in bone formation. Despite this, TGF-b is clearly important in the regulation of endochondral ossification and chondrogenesis, and its presence in normal fracture healing suggests that this factor plays a role in the normal repair process. However its actions are complex and not yet fully understood.

Bone Morphogenic Proteins (BMPS)

The ability of demineralised bone matrix (DBM) to induce bone formation was ascribed to a protein named “BMP”. Presently 15 BMPs have been identified. BMPs are able to induce bone formation in vivo and to promote osteoblast differentiation in vitro, also BMP-2 and BMP-7 stimulate osteoblastic differentiation. Thus BMPs play a local regulatory role in bone formation, remodelling and bone healing via stimulating the differentiation of bone marrow stromal cells. Interestingly mature cells seem to loose their responsiveness to BMPs. Also, differences in osteogenic effects of BMPs have been demonstrated, but the results are conflicting. However, individual BMPs may have different functions in endochondral ossification, demonstrated by the different temporal distribution of BMPs at the fracture site.

BMPs have been widely studied, and several studies show that the use of individual BMPs, combined with appropriate carriers, lead to bone repair in several species.

Insulin-Like Growth Factors (IGFs)

IGFs are growth factors synthesised by multiple tissues including bone. Two have been characterised: IGF-1 and IGF-2. IGF production in bone tissue is known to be stimulated by parathyroid hormone and growth hormone. Chondrocytes and osteoblasts possess receptors for growth hormone. The major effect of IGF in bone tissue is probably its potent effects on cartilage in the growth plate. It has been suggested that growth hormone controls longitudinal bone growth via the local stimulation of chondroblastic IGF production and IGF subsequently regulates chondrocyte mitosis and differentiation.

IGF-2 has been found less active than IGF-1 in its stimulation of growth cartilage undergoing endochondral ossification. Specific effects of IGFs are dependant on the cellular and hormonal environments.

IGF's are required for the proliferation of most cell types, and they promote cell survival by inhibiting programmed cell death. IGFs produced in bone cells are stored at a high concentration within the bone matrix where they regulate bone cells, resulting in increased osteoblast maturation. However in vivo, IGFs have had limited success as local stimulators of bone healing.

Platlet Derived Growth Factors (PDGFs)

The main effect of PDGF on bone cells is mitogenic. PDGF is also a powerful chemotactic factor for human osteoblasts. However PDGF has only a vague effect on metabolic functions of bone cells, but has an effect on other growth factors in bone formation. Several studies report that PDGF promotes cell replication, but inhibits osteoblast differentiation, maybe as a result of decreasing IGF mRNA in osteoprogenitor cells.

PDGF may act early on in the regenerative process to promote cell migration and cell replication, prior to the expression of factors that subsequently promote osteoblast differentiation.

Fibroblast Growth Factors (FGFs)

FGFs mainly have a proliferative effect on osteoblasts, and consequently they probably enhance bone formation by increasing the number of cells capable of bone formation. FGFs are present in normal fracture healing and have both mitogenic and angiogenic activities, promoting neovascularisation during the bone healing response. FGFs also have stimulatory effects through increasing cell synthesis of other growth factors.

Conversely it has been suggested that FGFs act as negative regulators of bone growth, but the results are conflicting. FGF signalling inhibits chondrocyte proliferation, and has an effect on chondrocyte differentiation.

However bFGF has been used to stimulate angiogenesis in molded bone graft, and FGFs may have possible future clinical use, especially since FGF's have both osteogenic and angiogenic properties.

Vascular Endothelial Growth Factor (VEGF)

VEGF mediated invasion by blood vessels is essential for coupling cartilage resorption with bone formation.

As in other physiological and pathological processes, neovascularisation in cartilage is finely modulated and is controlled by the balance of molecules with opposite potentials. Vascular invasion of cartilage is necessary for proper bone formation. The vasculate provides a conduit for the recruitment of the cell types involved in cartilage resorption and bone deposition and provides the signals necessary for normal bone morphogenesis.

Chondrocytes are able to synthesise both angiogenesis inhibitors and stimulators, depending on their culture conditions and state of differentiation. Resting and proliferative chondrocytes have strong anti-angiogenic effects, e.g. troponin I. Hypertrophic chondrocytes in vitro elicit neovascularisation, via angiogenic factors such as transferrin (a major angiogenic factor), bFGF and VEGF. VEGF produced by hypertrophic chondrocytes recruits endothelial cells and thus induces and maintains blood vessels (angiogenesis) during, endochondral bone formation.

Inactivation of VEGF results in nearly complete suppression of blood vessel invasion, and the delayed apoptosis of terminal hypertrophic chondrocytes. Thus VEGF-mediated blood vessel invasion is essential for coupling hypertrophic cartilage resorption with bone formation. Hypertrophic cartilage resorption, in turn, may potentiate the angiogenic process by degrading the ECM and increasing the bioavailability of VEGF and other mediators. In addition, exposure of endothelial cells to VEGF may trigger signalling cascades that lead to production of cytokines and proteinases and other mediators that then influence chondrocytes, chondroclasts and osteoblasts. VEGF is also chemotactic for cultured osteoblasts. Thus VEGF has a wider role outside neovascularisation, that is still being elucidated.

Other Growth Factors

Endochondral bone formation and fracture healing processes are well regulated at a biochemical level. These processes involve complex interactions between many local and systemic regulatory factors. In fact a very large cytokine network provides for bone development and allows bone integrity to be conserved during life. The cytokines which have effects on bone cells can be divided into the interleukins (IL-1, IL-3, IL-4, IL-6, IL-10, IL-11), the colony stimulating factors (GM-CSF, G-CSF, M-CSF) and tumour necrosis factor, TNF. These factors can be produced by osteoblasts and are probably involved in osteoblast-osteoclast interactions.

As the complex cellular interactions involved in the bone formation process are further elucidated and molecular techniques develop, the number of growth factors involved increases and their complex roles are realised. A variety of growth factors including IGF-1, IGF-II, bFGF, and PDGF have been assessed for their effects on osteoblast survival on their own, or together. They were unable to achieve 100% osteoblast survival, suggesting that either unidentified growth factors or ECM components maybe also responsible for promoting osteoblast survival.

Connective tissue growth factor-like (CTGF-L) is an example of a recently characterised bone growth factor. Its highly selective expression is suggestive of a selective role for CTGF-L in the control of bone formation. Also the cartilage-derived growth promoting factors chondromodulin-I and II have been shown to stimulate osteoblast proliferation.

Bone Grafts and Bone Graft Substitutes

When considering the experimental enhancement of bone healing and formation, three principles exist:
Osteoinduction—this is the formation of new bone via the stimulation of osteogenic precursor/stem cells. These cells differentiate and form new bone. (Osteoinduction is synonymous with “osseoinduction”).
Osteoconduction— this is enhanced bone formation due to a favourable structural environment where bone is formed.
Osteogenesis— bone formation can be stimulated by modulations of the natural biochemical processes that initiate and maintain bone formation during a healing response.
   Bone Grafts
   Autogenous Bone Graft (Autograft)

After autogenous bone grafting, bone formation is accomplished by osteoinduction via both a cell mediated mechanism, which occurs with viable precursor cells from the implant bone marrow stroma, and osteoinductive growth factors released from the bone matrix. Also other bone matrix derived growth factors participate in activation and maintenance of cellular processes during bone formation and healing. This adds to the osteogenic properties of autogenous bone graft.

Autogenous bone, recovered from the patient's own illac crest, is generally considered the gold standard of bone grafting material. However, due to limited autograft quantities, donor site morbidity, post-operative pain and other problems including variable biological performances there is a clear need for an alternative.

Allogenous Bone Graft (Allograft)

Donor allogenous demineralised bone matrix (DBM) has become widely accepted as a bone graft substitute in clinical practice. However because allografts are procured from humans, the transmission of disease from donor to the recipient is a concern. Of principle concern is the transmission of HIV, hepatitis B virus and hepatitis C virus.

Osteoinduction by DBM has been well, but incorrectly, documented. The in vivo osteoinduction properties of allograft DBM has been questioned and it has been shown that the new bone is actually generated by osteoconduction rather than osteoinduction. Allograft DBM and mineralised allograft bone acts as a scaffold on which existing bone grows, rather than the de novo differentiation of bone cells, independent of pre-existing bone.

Lack of osteoinductivity of allograft DBM has been accounted for by the processing and sterilisation procedures related to commercially available DBM. Mineralised allografts have a complex architecture that are potential reservoirs for viruses and infection. Therefore, although allograft is valuable, it is not without its problems. The abundant use of allograft, which has led to a shortage, is due actually to the lack of a safe, abundant alternative rather than its success.

Bone Graft Substitutes

Currently, two divergent pathways are being investigated in the field of bone healing. One pathway is the mimicry of the mineral phase of bone. The other pathway involves the creation of composite materials containing growth factors in specially designed carrier matrices, in an attempt to create a material that will surpass autograft in osteogenic potential.

Coralline Hydroxyapatite Porous Implants

Bone formation requires a physical structure to which osteoblasts can adhere. It also requires vascularisation. Therefore the concept of using porous coral skeletons as templates for bone graft substitutes was conceived. Capillaries, perivascular tissues and osteoprogenitor cells can migrate into porous spaces and incorporate the porous structure with newly formed bone.

Bone forms via intramembranous ossification within the pores of the coralline hydroxyapatite porous implants. For bone ingrowth to occur the implant must be rigidly stabilised and in close opposition to host bone. Although the bone is remodelled over time, the implant is not significantly remodelled due to the insoluble, inert structure of crystalline hydroxyapatite.

This non-remodelled implant compromises the ultimate mechanical strength of the bone in some clinical situations. The poor initial strength and handling characteristics are also a disadvantage. Also, any new bone is slight and is formed through the process of osseoconduction rather than osseoinduction.

Tricalcium Phosphate (TCP)

Tricalcium phosphate (TCP) porous implants, which are soluble stimulate osteoclastic remodelling, which, in turn, results in new bone formation within the resorbed regions of the implant. In bulk, the rapid dissolution of TCP can be used in improving hydroxyapatite implants. TCP is sintered into hydroxyapatite structures resulting in improved ingrowth of bone and new bone formation.

Bone Growth Factor Carriers

Several studies show that the use of individual BMPs (e.g. BMP-2/3/7), combined with appropriate carriers, leads to bone repair in several species. One such carrier is collagen prepared by complete demineralisation of bovine trabecular bone. Collagen is an ideal carrier for BMPs and other growth factors and appears to provide an optimal template for vascular invasion, deposition of mineral, and initiation of active remodelling. However, because of concerns about possible pathogen transmission of allogenic or xenogenic (taken from different species usually of bovine origin) materials, the search for synthetic carriers has been prompted. Furthermore, very recent studies have shown that BMP-7 is not, in fact, a bone growth factor but is angiogenic instead. This would explain why it seems necessary to supply huge (30 plus milligrammes) quantities of BMP-7 in order to elicit tiny bone growth responses in vivo.

Potential synthetic carriers include porous ceramics, bioactive glasses and polymers. Porous ceramics can be classified as non-resorbable such as synthetic hydroxyapatite, or resorbable, such as tricalcium phosphate (TCP) and calcium sulfate (Ca/S). Polymers used as carriers of BMP comprise of various forms of polyglycolic acid (PGA) and polylactic acid (PLA). Bone and cartilage growth on such implants is not optimal and offer little osteoconductive potential and no osseoinductive potential. These polymers slowly degrade by hydrolysis and offer a possibility of controlled growth factor release. However, a large proportion of the proteins do not retain activity after release from the polymer carrier.

The development of an optimal biosynthetic matrix has yet to be achieved. Biosynthetic matrices do not compare favourably to the performance of biologic materials used as osteoconductive implants, such as bovine collagen. So, despite the concern of pathogen transmission bovine collagen and allograft collagen is still being used.

The first human pilot studies have been performed using BMP-2/7 on a collagen carrier for bone reconstruction. However, there was a large variation among the responses of individual patients especially those with maxillary or mandibular atrophy. The inconsistent results from these clinical pilot studies suggest that certain factors negatively affect the BMP dependent bone induction process in humans, or that BMP-2/7 may have no true osseoinductive activity.

Since BMPs are not exclusive in promoting bone formation, other growth factors are also being investigated as possible in vivo osteoinducers. TGF-B may be useful in the enhancement of bone ingrowth and thus improve the mechanical fixation of non-cemented implants. A combination of PDGF and IGF in a gel formulation has been used to successively stimulate bony ingrowth into dental titanium implants.

Just as two factors may be expected to exert a more profound effect than one, so may three or more regulatory factors delivered at appropriate times be expected to have a greater effect than one factor alone. However, so far the study of in vivo enhancement of bone healing has been limited, to mainly the use of one growth factor.

Bone healing involves a well orchestrated series of events, under the tight control of several growth factors. It is unlikely that a single factor acts in is temporal or spatial isolation from the others. Even a factor with little evident effect in isolation may be highly significant as a modulator of some other regulatory factors. The recognition of such growth factor interaction has received very limited attention in the field of fracture repair.

As a consequence of the consensus above being reached—that autogenous bone graft material containing living human cells provides the only real source of osseoinductive activity—some researchers have extended the use of living marrow cell grafting to give a novel approach to bone repair and regeneration. Studies have shown that autologous mesenchymal cells, derived from the marrow, can be reimplanted into cranial defect and other skeletal defect sites, after cell number expansion in vitro, to induce new bone formation. This work, once again supports the principle of autologous grafting of living bone (osteoblastic) precursor/forming cells as a method of orchestrating the induction of new bone growth by intramembranous and endochondral ossification.

However, as a therapy, it does have its drawbacks. The use of autologous cells for patient treatment on a worlwide scale would be a particularly arduous and expensive task. It would be necessary to sample each patient's marrow for stem cells, and to then grow them in culture for several weeks/months under sterile conditions prior to returning them to the patient for their bone repair/regeneration. It also, would not overcome the morbidity problems associated with patient sampling, nor the potential variation in the quality of the material being used. Furthermore, such an approach would not have value in the immediate treatment of trauma cases, which is often required, particularly after road traffic accidents.

### SUMMARY OF THE INVENTION

The claimed invention is directed to an extracellular material obtained from skeletal cells, which material has osseoinductive bone repair/regeneration activity in vivo and is in freeze-dried (lyophilyzed) form. This material may be essentially cell free, in particular it may be essentially free of DNA from said cells. Also, the material may be essentially free of cell debris. The material may be in essentially isolated and purified form.

The material of this invention may be obtained from various sources, for example from cartilage cells and from chondrocyte cells. In one embodiment the material is obtained from hypertrophic cartilage cells, in another the material is obtained from immortalised hypertrophic chondrocyte cells. The cells from which the material is obtained may be cells from any eukaryote having skeletal cells, but are preferably human cells, for example a human cell line.

The material of this invention may contain a mixture of: (1) one or more cytokine; (2) one or more growth factor; and (3) one or more collagen.

Also part of this invention is a therapeutic composition which comprises or consists of an active ingredient which is an effective amount of any of the materials of this invention, i.e. any osseoinductive material of this invention. Such a therapeutic composition according preferably includes a physiologically acceptable excipient and/or adjuvant and/or carrier. Such components are well known in the art. Another active ingredient may be included.

Provided that the material of the invention has been produced in lyophilyzed form, any composition of this invention may be provided subsequently in frozen form, or in frozen-thawed form, or in freeze-dried (lyophilyzed) form.

Also part of this invention is a method for producing osseoinductive extracellular

material from skeletal cells which method comprises or consists of the steps of:

(1) culturing skeletal cells in a suitable culture medium;
(2) harvesting extracellular material produced by said cultured cells; and optionally
isolating and/or purifying said harvested material; and
(3) lyphilyzing said material.

Also part of this invention (where legally permissible) is a method of treating a patient (human or other animal) requiring bone repair/regeneration, which involves administering to said patient an osseoinductive amount of a material or composition of this invention.

This invention also provides for the use of an osseoinductive material from skeletal cells for the manufacture of a therapeutic agent for the treatment of a condition requiring bone repair/regeneration.

### DETAILED DESCRIPTION OF THE INVENTION

An optional additional step is that of (4) adding a physiologically acceptable excipient and/or adjuvant and/or carrier, to form a therapeutic composition. Materials and compositions produced by these methods are also part of this invention.

Our understanding of the basic biology of the endochondral ossification process in natural skeletal growth as well as in fracture repair recognises the importance of hypertrophic cartilage chondrocytes as part of the bone forming process. The method of the invention involves using human hypertrophic cartilage derived cells as a source of a therapeutic biological material. Such material has never been isolated previously, nor have such cells been considered as a potential source material for the treatment of patients requiring bone repair/regeneration.

It is known from WO 96/18728 that it is possible to isolate and immortalise human hypertrophic chondrocyte derived cells which can be used to generate large homogeneous populations of cells under laboratory conditions.

The invention provides a therapeutic biological material harvested from hypertrophic cartilage chondrocyte (and other skeletal stem/progenitor/precursor) derived cells, and provides for its use for the repair and regeneration of skeletal defects. The material of the invention comprises a highly complex mix of cytokines, growth factors, matrix and other proteins which provide for osseoinductive activity. The invention also provides for a surprising and interesting finding that the freezing and drying of the material of the invention prior to use does not compromise its biological/osseoinductive activity, unlike such treatments of bone allograft material which destroy the biological/osseoinductive activity of the allograft sample.

It is an advantage of the invention that, despite only surprisingly small amounts of specific growth factors being present in the material, there is clear osseoinductive activity of the material if it is placed in the skeleton. Also, the invention demonstrates that the osseoinductive activity remains despite there being a surprisingly low level of type X collagen in the material. This is wholly unexpected in light of the substantial amount of type X collagen seen in growing bones that are undergoing endochondral ossification, and the amount of type X collagen in fracture callus, which is undergoing intramembranous and endochondral ossification, along with living hypertrophic chondrocytes.

It is a further advantage of the material of the invention that it can be produced as a cell-free therapeutic product. This can allow it to be physiologically acceptable for medical purposes and can allow it to more easily achieve Regulatory Approval than would be possible for a therapeutic biological product which comprises or consists of cells (whether dead or alive). A crude extract of the material of the invention can be purified by methods known to those in the art. Cell debris can be removed by suitable filtration. DNA can be removed or degraded by use of a suitable DNAase.

Furthermore, the method of the invention does not rely on the need for living cells to conduct the process of bone repair/regeneration, nor does it rely on trabecular bone/bone marrow as being the material from which the cells are sourced.

### DESCRIPTION OF THE FIGURES

FIG. 1 is a 2-D gel electrophoresis analysis of “Skeletex” material according to the invention.

FIG. 2 is a 2-D gel electrophoresis analysis of Foetal Calf Serum, as a control comparison.

FIG. 3 shows a dose-related expression of osteocalcin by rat marrow cells incubated with “Skeletex” material according to the invention.

FIG. 4 shows extensive trabecular vascularised bone formation in rat femur treated with “Skeletex” material according to the invention. Product applied into a hole drilled into the rat femur. Hole plugged with wax. Sections taken after 2 weeks. A) shows extensive, trabecular, vascularied bone induced Skeletex™. B) shows space left by wax plug used to keep Skeletex™ in place. C) shows rat femur cross section.

FIG. 5 shows a control treatment of rat femur, for comparison. A) shows very limited bond formation without Skeletex™. B) shows area of wax plug. C) shows bone marrow.

FIG. 6 a VEGF standard curve, for use in the assay of Example 6.

FIG. 7: The graphic shows a schematic representation of constructs transfected into PA317 packaging cells to produce the PA317 cmv tsT cell line which produces retroviral particles containing the large T antigen tsA58 mutant.

FIGS. 8-22 show the results of Example 1: Charts 1-15.

The invention is described but not limited by way of the following examples:

Immortalisation of Human Skeletal Cells

The immortalisation of human skeletal cells has been described previously in copending applications (see WO 96/18728, for example). The process of cell immortalisation can be through genetic means, such as genetically engineering the human skeletal cells with an immortalising gene—for example: simian virus-40 large T antigen or any other replication enhancing viral oncogene; or eukaryotic oncogene such as mammalian Harvey-ras, or Kirsten-ras, or N (neuroblastoma)-ras, or bcl-2, or c-myc; or a modified tumour suppressor gene such as p53 or ICE whereby the modification has enhanced the replication potential of the cell containing and expressing the gene. Alternatively the immortalisation might be through the addition of an immortalising agent(s), such as a growth factor(s) eg. EGF, FGF, VEGF, CDGF, IGF, IGFBP3, CTGF, BMP, TNF, TGF, or a cytokine or protease such as a MMP or inhibitor such as a TIMP; or an activating chemical such as retinoic acid, IBMX, or cAMP, cGMP etc. Also carcinogens such as benzine, chloroform, tar and extracts thereof may serve as immortalising agent(s).

A process of skeletal cell immortalisation is provided below, but is not limiting as any method of immortalisation—either genetic such as transduction or transfection or otherwise, or by any agent inducing cell immortalisation—is acceptable.

Preparation of Immortalising Vector

The SV40 large-T antigen mutant tsA58 encodes a thermolabile protein which is active at the permissive temperature (33° C.) and inactive at the non-permissive temperature (39° C.). Using this mutant SV40-T to immortalise cells, the return to a non-immortalised state can be obtained by incubating the cells at the non-permissive temperature. This construct, which also contains a neomycin-resistance marker, has been cloned into a retroviral vector, which is a highly efficient means of transferring genes into cells. Retroviruses are RNA viruses; when it penetrates a cell, the viral RNA is reverse transcribed to DNA, and the DNA enters the nucleus and integrates randomly into a chromosome. Progeny viruses are formed, which leave the cell by budding from the cell membrane. The viral genome contains two types of information, which can be classified as cis and trans. The trans functions are the viral proteins such as the polymerase and the envelope glycoproteins. The cis functions are the various signals such as the promoter and enhancer sequences required for initiation of RNA transcription, the sequences which direct the integration of the viral genome into the chromosome of the infected cell, and the encapsidation signal (y) required for virus packaging. To construct a virus that can be used as a vector, the cis functions must be retained while the trans functions can be replaced by the gene of interest. Hence, a replication-defective retrovirus missing its own genes is created. The trans functions can be supplied by a “helper virus”. Thus the retroviral vector carrying the gene of interest can be assembled into a virion, exit from the cell, infect a target cell and, through the cis functions retained in the vector, the foreign gene is transferred into a chromosome of the cell as if it were a viral gene. In the laboratory, this process takes place in two steps. Initially, the portions of the retroviral DNA carrying the cis functions are combined with the gene of interest. Subsequently, this vector DNA is transfected into a packaging cell line, which contains a helper virus. The vector DNA is then transcribed into viral genomic RNA, which is encapsidated into a retroviral virion and secreted into the medium. The recombinant virus can transfect a target cell and integrate into its genome, and because the viral RNA does not contain the trans functions, it cannot replicate. It is possible to isolate the cells that have taken up the vector DNA by use of a selectable marker present in the vector, such as neomycin.

A problem which arises when retroviral vectors are used is the possibility that replication-competent viruses could form and that the proliferation of these viruses would lead to multiple integrations into the genome. To avoid such complications, packaging cell lines, such as PA317 have been constructed in which the retroviral sequences in the helper virus have been additionally mutated. These mutations have included deletions in the 3′ long terminal repeat (LTR) of the helper virus, and additional deletions of portions of the 5′ LTR as well.

Frozen vials of PA317 cmv tsT cells were thawed rapidly by placing the vial in a beaker of water preheated to 37° C. Once thawed, the vial was sprayed with 70% ethanol and the cells were transferred to a sterile universal tube containing 10 ml a-MEM growth medium. The cells were centrifuged at 500 g for 5 minutes at 4° C. and the cell pellet was resuspended in 6 ml of fresh growth medium and seeded in a T25 culture flask and incubated at 33° C. in a humidified atmosphere of 95% air/5% CO₂. Before reaching confluence the cells were passaged to a T75 culture flask. During the period that the cells took to reach confluence, the conditioned medium was removed on a daily basis and replaced with fresh growth medium. The collected conditioned medium was filtered through a 0.22 mm acrodisc filter to remove any cell debris and the resultant solution was aliquoted and snap frozen by immersion in liquid nitrogen for 5 minutes. The frozen aliquots were stored at −80° C.

Immortalisation of Human Skeletal Cells

Cells, derived either from human adult bone or cartilage, or marrow, or trabecular bone biopsies comprising either foetally-derived, newborn, pre-adolescent, or adolescent derived cells were grown to 80% confluence in a tissue culture flask. They were then exposed to the filtered retrovirus-containing medium mixed in a 1:1 ratio with growth medium and polybrene at 8 mg/ml. The container was incubated at 33° C. with a humidified atmosphere of 95% air/5% CO₂ for two hours in order to allow the retrovirus to transduce the cells. Following incubation, the medium was removed and the cells were washed three times with PBS (1×) and fresh growth medium was added to the container, which was put back in the incubator at 33° C. When the cells had reached confluence, G418 (geneticin 500 mg/m) was added to the medium and the cells were kept at 33° C. for between 10 and 15 days. During the selection period the growth medium was changed every three days. G418-resistant colonies emerged which represented individual clones of transduced skeletally-derived cells.

Ring Cloning of G418-Resistant Immortalised Human Skeletal Cells.

The G418-resistant colonies were marked for ring cloning by first identifying them in the flasks by inverted microscopy and then marking the position of each colony with a pen on the bottom surface of the tissue culture container. Only colonies that were well-demarcated were marked for cloning so as to avoid mixing of colonies. To facilitate the removal of the colonies from the flask, the top was cut away using a heated scalpel blade. Once the top was off, the medium was removed and the cells were washed three times with PBS (1×). The third wash was left in the flask to avoid drying while preparing the cloning rings. The top 5-10 mm of a 1 ml Gilson's pipette tip was cut off using a scalpel blade heated over a bunsen burner. The smooth uncut side of each ring was smeared with sterile autoclaved vacuum grease and placed on autoclaved tin-foil paper until required. The PBS (1×) was then removed from the flask and using the marked circles as the position of each colony, the cloning rings were placed on top of them and gently pressed down to ensure that the ring was sealed to the surface with the vacuum grease. After the cloning rings were positioned, 50-100 ml of trypsin/EDTA was placed into each ring and left for 1-2 minutes to allow the cells to detach from the tissue culture flask. The cells in trypsin were also pipetted up and down to help detachment of the cells from the surface and subsequently the detached cells were transferred to a separate well of a 48-well tissue culture plate, in 200 ml of fresh growth medium. Once the sample cells had descended to the bottom of the well, the top 100 ml of medium was removed carefully and replaced with fresh medium containing G418 (500 mg/ml).

The plate was transferred to a 33° C. incubator with a humidified atmosphere of 95% air/5% CO₂ and left for 24 hours to allow the cells to attach to the bottom surface of the culture well. After this period the medium was removed and replaced with fresh growth medium containing G418 at the same concentration and the cells returned to the 33° C. incubator with a humidified atmosphere of 95% air/5% CO₂ until the cells were nearly confluent; at this point they were removed with trypsin/EDTA and transferred to wells of a 24-well plate to reach confluence again. The cells were further transferred to bigger tissue culture flasks until enough cells had been obtained for each clone to allow cryopreservation.

The Growth of Human Fetal Femur Immortalised Hypertrophic Chondrocytes

Materials

Medium

MEM alpha medium (1×), liquid, with L-glutamine, ribonucleosides, deoxyribonucleosides was purchased from Life Technologies (Paisley, UK, catalogue no. 22571-020).

Fetal Bovine Serum (FBS)

FBS with origin from Australia was purchased from Life Technologies (Paisley, UK, catalogue no. 10099-141).

Glutamine

L-glutamine, 200 mM (100×), liquid, was purchased from Life Technologies (Paisley, UK, catalogue no. 25030-024).

Penicillin/Streptomycin (P/S)

P/S: 10,000 u/ml penicillin, 10,000 g/ml streptomycin was purchased from Life Technologies (Paisley, UK, catalogue no. 15140-122).

Non Essential Amino Acids (NEAA)

NEAA (100×), liquid, without L-glutamine was purchased from Life Technologies (Paisley, UK, catalogue no. 11140-035).

Trypsin/EDTA

Trypsin/EDTA (IX) 0.05%/0.02% w/v, liquid, was purchased from Life Technologies (Paisley, UK, catalogue no. 25300-054).

Dulbecco's Phosphate Buffered Saline (PBS)

PBS (10×), liquid w/o calcium and magnesium was purchased from Life Technologies (Paisley, UK, catalogue no. 14200-067).

Tissue Culture Flasks

Filter cap flasks with culture area of 25 and 75 cm² were purchased from Nunc is plasticware Life Technologies (Paisley, UK, catalogue no. 156367A and 156499A respectively).

Pipettes

Sterile pipettes of 5 ml and 10 ml were obtained from Sterilin LTD. (Middlesex, UK, catalogue no. 47105 and 47110 respectively).

Centrifuge Tubes

Sterile 50 ml disposable centrifuge tubes were purchased from Corning (catalogue no. 430291).

Methods

Solutions

Growth Medium (500 ml Solution)

435 ml of a-MEM, 50 ml of heat-inactivated FBS, 5 ml of 100× L-glutamine solution, 5 ml of 100× MEM NEAA, 5 ml of 100× P/S solution. After preparation, the solution is kept at 4° C. for no more than two weeks.

Phosphate Buffered Saline (PBS)

1× PBS solution is prepared by dilution Dulbecco's Ca²⁺—Mg²⁺-free phosphate buffered saline 10× concentrate with autoclaved ddH₂O. To achieve sterility the solution is autoclaved. It is stored at room temperature.

Fetal Bovine Serum (FBS)

Batches of FBS are heat-inactivated by placing the containers in a water bath at 56° C. for 30 minutes, aliquoted and stored at −20° C.

Freezing Medium

To 20 ml of normal medium, 5 ml of DMSO and 25 ml of FBS are added. It is stored at 4° C. for up to 2 months.

Breaking Out Cryopreserved Cells

When cell are required, the cryotube is placed in the hood at room temperature.

When the cells are fully thawed, they are immediately transferred to a universal containing 10 ml of growth medium, which was taken out of the fridge and left at room temperature for at least 15-20 minutes.

The cell suspension is centrifuged at 500 g for 5 minutes at room temperature.

The cell pellet is resuspended in 6 ml fresh growth medium, transferred in a T25 culture flask and incubated at 33° C. in a humidified atmosphere of 95% air/5% CO₂.

The medium is changed after 24 hours to remove cell debris.

Detachment of Cells from Tissue Culture-Treated Plastic Flasks

Monolayers of cultured cells are washed three times with sterile PBS (1×), which was taken out of the fridge and left at room temperature for 15-20 minutes.

Detachment of the cells is induced by addition of enough trypsin/EDTA solution to cover the surface on which the cells are growing.

The cells are then incubated at 33° C. for approximately 1 minutes, with cell detachment being monitored by phase contrast microscopy every 15-30 seconds.

If the detachment of cells is incomplete after 1-2 minutes, then the process is encouraged by gentle aspiration of the trypsin/EDTA solution across the flask, using a sterile pipette.

The growth medium is taken out of the fridge and left at room temperature for 15-20 minutes prior to use. 10 ml of growth medium are added to the cells and the suspension is centrifuged at 500 g for 5 minutes at room temperature.

The resultant pellet is resuspended in a known volume of fresh growth medium and dispersed into a single cell suspension by repeated aspiration through a sterile pipette.

The total number of cells of a 90-95% confluent flask of human fetal femur (HFF) hypertrophic chondrocyte cell line is split 1:4 when being passaged, therefore the appropriate volume of cells is transferred to the new culture flasks together with additional medium to cover the surface of the flask.

These are then transferred to a 33° C. incubator with a humidified atmosphere of 95% air/5% CO₂. The medium is changed twice a week.

Preparation of Cryopreserved Cells

Logarithmically-growing cells are detached with trypsin-EDTA using the method described above.

After centrifugation, the cell pellet is resuspended in freezing medium at a cell density of 2×10⁶ cell/ml.

The cell suspension is transferred to cryotubes in 1 ml volumes.

The sealed cryotubes are then placed at −80° C. for a minimum of 3 hours before being transferred to the liquid phase of a liquid nitrogen cylinder for storage.

Harvesting of Material Produced by Human Fetal Femur Immortalised Hypertrophic Chondrocytes

In the following description the harvested therapeutic biological material of the invention may be referred to by the term “matrix”. This matrix material is also referred to by the Trade Mark “SKELETEX”.

Matrix from HFFC13 cells can be usually harvested twice weekly ONLY when the cells are at least 50% confluent. The matrix from 3×75 cm² flasks, which have been seeded on a Monday, can be harvested on Thursday and subsequently on the following Monday prior to passaging.

NOTE: This schedule is NOT exact, and will depend upon the growth characteristics of the cells: ie. it may be possible to harvest matrix between the 3rd and 4th day after seeding. Also, passaging is carried out only when the cells are at 95% confluence (which can be between 7 to 9 days).

The growth medium is transferred to a universal and centrifuged at 1200 rpm for 5 minutes at room temperature.

The supernatant is discarded and the pellet of Skeletex is stored at −80° C. until required.

Freeze-Drying of SKELETEX

The pellet of Skeletex is thawed out at room temperature and transferred to a sterile centrifuge tube. Because of the small volume used (between 100-200 ml) the sample does not have to be frozen prior to being freezed-dryed.

The centrifuge tube is loaded on the centrifuge of the freeze-dryer and the process is started. Freeze-drying is a controllable method of dehydrating labile materials, often of biological origin, by desiccation under vacuum.

The centrifuge is placed in a vacuum chamber where a vacuum is created to reduce the air concentration above the product and encourage sublimation, and to make sure that the air leaking into the system is removed.

When the process is finished and the sample freezed-dryed (liophilised) it is stored at room temperature for up to 5 days. To avoid contact of the sample with atmosphere and consequently reabsorption of damp air into the product, laboratory film is wrapped around the top of the centrifuge tube.

Isolation of Rat Bone Marrow Cells (BMCs)

Materials

Dulbecco's Modified Eagle Medium (DMEM) (1×)

DMEM with 4500 ml/L D-glucose, non essential amino acids, without L-Glutamine and sodium pyruvate was purchased from Life Technologies (Paisley, UK, catalogue no. 10938-025).

Sodium Pyruvate 100 mM, Liquid

Sodium pyruvate was purchased from Life Technologies (Paisley, UK, catalogue no. 11360-039).

Penicillin/Streptomycin (P/S)

P/S: 10,000 u/ml penicillin, 110,000 μg/ml streptomycin was purchased from Life Technologies (Paisley, UK, catalogue no. 15140-122).

Fetal Bovine Serum (FBS)

FBS with origin from Australia was purchased from Life Technologies (Paisley, UK, catalogue no. 10099-141).

Glutamine

L-glutamine, 200 mM (100×), liquid, was purchased from Life Technologies (Paisley, UK, catalogue no. 25030-024).

Dexamethasone

Dexamethasone was purchased form Sigma (Dorset, UK, catalogue no. D8893)

b-glycerophosphate

b-glycerophosphate was purchased from Sigma (Dorset, UK, catalogue no.

G9891)

Ascorbic Acid

L-ascorbic acid was purchased from Sigma (Dorset, UK, catalogue no. A4034)

Tissue Culture Flasks

Filter cap flasks with culture area of 25 cm² were purchased from Nunc plasticware Life Technologies (Paisley, UK, catalogue no. 156367A).

Pipettes

Sterile pipettes of 5 ml and 10 ml were obtained from Sterilin LTD. (Middlesex, UK, catalogue no. 47105 and 47110 respectively).

Centrifuge Tubes

Sterile 50 ml disposable centrifuge tubes were purchased from Corning (catalogue no. 430291).

Methods

Solutions

Bone Juice (500 ml Solution) for Calcifying Cultures

410 ml of DMEM, 70 ml heat-inactivated FBS, 5 ml of 100×L-glutamine solution, 5 ml Pen-Strep, 5 ml of 100 mM pyruvate solution, 5 ml of a 1M beta-glycerophosphate solution, 0.5 ml 10⁻⁵ M dexamethasone, final concentration 10⁻⁸ M, 50 mg/ml ascorbic acid final concentration.

Beta-glycerophosphate, dexamethasone and ascorbic acid are first added to 10 ml DMEM, the solution is then filtered through a 0.22 mm acrodisc filter into the final medium. It is stored at 4° C. for 1 week. Ascorbic acid must be renewed at every medium change.

Fetal Bovine Serum (FBS)

Batches of FBS are heat-inactivated by placing the containers in a water bath at 56° C. for 30 minutes, aliquoted and stored at −20° C.

Kill rat by cervical dislocation or any other schedule 1 method.

Remove the back legs at the hip joint using a pair of sterile scissors.

Remove as much soft tissue as possible and carry to cell lab in a sterile container (e.g. petri dish).

Under sterile conditions remove all the soft tissue. Cut the leg at the knee joint and ankle joint. Remove growth plates on both tibia and femur. Cut the femur a little below the hip joint, where the marrow ends and the tibia at the ankle joint (have two bones per leg) using a bone cutter or a saw

Make a hole using a 18 g needle, on the uncut side of the bone.

Place the cut bones, cut side down, in inserts in eppendorf tubes and spin at 2000 rpm for 2 minutes to flush bmcs out. Remove the bones and the inserts.

Resuspend the bmc in 0.5 ml bone juice and then make them up to 5 ml per bone in bone juice.

Make a single cell suspension by forcing the cells through a 18 g needle.

Make up to 10 ml per bone with bone juice (about 2×10⁶ mononuclear cells/ml).

Seed cells according to experiment being carried out.

Add bone juice and drugs as necessary and leave for 5 days at 37° C., 95% air/5% CO₂.

On the 5th day change medium, and from then on twice weekly until large calcified colonies have formed (between 12-18 days).

Remove 0.1-1.0 ml of used medium from each flask at each medium change, for each experiment, and store frozen at minus 20° C.-80° C. for subsequent osteocalcin assay.

To stop the cultures, rince once with PBS, fix with ethanol for about 5 minutes and then wash with tap water.

Fibroblastic Colony Forming Unit (CFU-f) Assay.

1. Seed BMC cells at a density of approximately 2×10⁶ per 10 cm petri dish (0.5 ml of cell suspension from see above). This can be varied according to the experiment being carried out.

2. Add 10 ml BJ, add drugs as necessary and leave for 5 d.

3. On the 5^{th} day, change medium; suck off old medium using the vacuum apparatus and replace with 10 ml BJ.

4. From the 5^{th} day on, change medium twice weekly (usually Monday and Thursday) for BJ+.

5. Inspect the colonies daily.

6. When ready i.e. large and calcified, usually between 12 and 18 days, the cultures can be stopped.

7. To stop the cultures, rinse once with PBS, fix with ethanol for about 5 min and then wash with tap water.

Alkaline Phosphatase Staining of CFU-f Cultures.

1. Make up APase buffer. Tris (20 mM, pH 8.5) containing naphthol phosphate AS-BI (50 ug/ml) and fast red (1 mg/ml).

2. Ensure that all petri dishes have been previously fixed and washed with tap water.

3. Add 5 ml APase buffer to each petri dish and shake at room temperature for 30 min.

4. Wash with tap water and allow to dry.

5. Photograph dishes.

Destain the dishes by shaking overnight with IMS and then washing with tap water.

Calcium Staining of CFU-f

1. Make up calcium stain. Alizarin red (1 mg/ml) in distilled water adjusted to pH 5.5 with ammonium hydroxide.

2. Ensure that all petri dishes have been previously fixed, destained and washed with tap water.

3. Add 5 ml calcium stain to each 10 cm petri dish and shake at room temperature for 30 min.

4. Wash the dishes well with tap water and allow to dry.

5. Photograph the dishes.

6. Destain the dishes with 5% perchloric acid and wash well with tap water.

Collagen Staining of CFU-f

1. Make up saturated picric acid by leaving an excess of picric acid over distilled water for 18 h. Decant the saturated picric acid.

2. Make up collagen stain. Sirius red (1 mg/ml) in saturated picric acid.

3. Ensure that all petri dishes have been fixed, destained and washed with tap water.

4. Add 5 ml of collagen stain to each petri dish and shake at room temperature for 18 h.

5. Wash with tap water until no more red colouring is eluted.

6. Photograph dishes.

7. Destain with 0.2 M sodium hydroxide/methanol (50:50).

Total Colony Staining of CFU-f

1. Make up borate buffer concentrate. Boric acid (1 M) adjusted to pH 8.8 with 0.2 N sodium hydroxide.

2. Make 10 mM borate buffer solution (10 ml concentrate in 1 litre distilled water) and check pH.

3. Make methylene blue solution. Methylene blue (1 mg/ml) in 10 mM borate buffer.

4. Ensure that all petri dishes have been fixed, destained and washed with tap water.

5. Add 5 ml methylene blue solution to each petri dish and shake at room temperature for 30 min.

6. Wash the dishes with tap water until no more dye is eluted.

7. Photograph the dishes.

The dishes can be destained if necessary with 1% HCl in ethanol.

Rat Osteocalcin Elisa

Measurement of Rat Osteocalcin from Rat Bone Marrow cell culture medium

Collect 1 ml of culture medium prior to replenishing the medium on day 5, 9, and day 13. Store samples at minus 80° C. until analysis.

Osteocalcin is measured by ELISA kit. (Catalogue No BT-490) Supplier IDS Ltd, Bolden Business Park, Bolden Tyne and Wear, NE35 9PD.

Store all reagents at 4° C. up to six months except where noted. NB Donkey anti-goat IgG peroxidase Conjugate store at minus 20° C.

All reagents and samples must be at room temperature just prior to analysis.

Assay 100 ul of sample, standards and QC's in duplicates.

Osteocalcin concentration is proportional to colour change compared with reagent standards.

Further information is supplied in the manufacturer's handbook.

In Vitro Bioassay of Hypertrophic-Like Chondrocyte Cells and Their Products

### EXAMPLE 1

An immortalised cell line identified as a human hypertrophic chondrocyte cell line was used as the example in this study (ref: WO 96/18728).

Experiment to Determine the In Vitro Osteoinductive Properties of Hypertrophic Chondrocytes on Rat Bone Marrow Cells, Using a CFU-f Assay.

Using the cell culture techniques described in the materials and methods sections above the following experiment was set up. To enable statistical analysis, each treatment group contained 3-4 petri dishes (10 cm diameter); but, where possible, 4 dishes were used to allow for validation of results if one dish became infected and thus void.

Control group. Bone Marrow Cells (Bmcs).

To 4 petri dishes, 10 ml of bone juice was added to 0.5 ml of rat BMC suspension (5×10⁵ cells) and incubated at 37° C. and 5% CO₂. No further additions were made and the medium was changed as specified in the method.

Treatment Group 1. Hypertrophic Chondrocytes—Mitomycin-C Treated Plus BMCs

3 petri dishes were seeded with 1.4×10⁶ cells. This is approximately at 40% confluence. 10 ml of growth medium was added to each dish and they were incubated at 33° C. and 5% CO₂ for 18 hours. The next day they were treated with 10 ul of mitomycin-C (1 mg/ml). Mitomycin-C is a DNA chelating agent which, once cells are treated with it, blocks all cell replication permanently. This enables the expression and accumulation of factors such as cytokines, growth factors and matrix proteins, but without any further cell replication and with the subsequent death of the treated cells.

Once the cells were treated and washed with PBS, 10 ml bone juice was added to 5×10⁵ rat BMCs and the resulting suspension added to each of the petri dishes. They were incubated at 37° C. and 5% CO₂. And the medium changed as specified.

Treatment Group 2. Hypertrophic Chondrocytes—MitC Treated, Grown for 1 week+BMCs.

4 petri dishes were seeded with 1.4×10⁶ cells. 10 ml of growth medium was added to each dish and they were incubated at 33° C. and 5% CO₂ for 18 hours. The next day they were treated with 10 ul of mitomycin-C (1 mg/ml) as described in the method. Once the cells were treated and washed with PBS, 10 ml growth medium was added to each of the petri dishes. They were incubated at 33° C. and 5% CO₂ for 1 week. And the medium changed twice during the week.

The cells were incubated for a week to allow them to lay down an extracellular matrix. The cells were plated a week before the date the experiment (addition of BMCs) was due. This was done to ensure all treatment groups were plated with the same number of rat BMCs, from the same suspension.

Treatment Group 3. Hypertrophic Chondrocytes—Freeze Thawed+BMCs.

3 petri dishes were seeded with 1.4×10⁶ hypertrophic chondrocytes. 10 mi of growth medium was added to each dish and they were incubated at 33° C. and 5% CO₂ for 18 hours. The next day they were freeze—thawed as described in the method, although in this instance, due to unforeseen circumstances, they were kept frozen for 18 hours, instead of 1 hour. Once the cells were thawed and washed with PBS, 10 ml bone juice was added to 5×10⁵ rat BMCs and the resulting suspension added to each of the 60 petri dishes. They were incubated at 37° C. and 5% CO₂. And the medium changed as specified.

After 1 week the growth medium was removed and the cells were washed with PBS. 10 ml bone juice was added to 5×10⁵ rat BMCs and the resulting suspension added to each of the petri dishes. They were incubated at 37° C. and 5% CO₂. And the medium changed as specified.

Treatment Group 4. Hypertrophic Chondrocytes—MitC Treated, Grown for 1 week Freeze-Thawed+BMCs.

4 petri dishes were seeded with 1.4×10⁶ hypertrophic chondrocytes. 10 mil of growth medium was added to each dish and they were incubated at 33° C. and 5% CO₂ for 18 hours. The next day they were treated with 10 ul of mitomycin-C (1 mg/ml) as described in the method. Once the cells were treated and washed with PBS, 10 ml growth medium was added to each of the petri dishes. They were incubated at 33° C. and 5% CO₂ for 1 week, and the medium changed twice during the week.

After 1 week the growth medium was removed and the cells were washed with PBS. The cells were then freeze-thawed as described for the other frozen-thawed cells. Once they were washed with PBS, 10 ml bone juice was added to 5×10⁵ rat BMCs and the resulting suspension added to each of the petri dishes. They were then incubated at 37° C. and 5% CO₂ and the medium changed as specified.

This treatment of the cells was performed to provide a group of petri dishes with a layer of hypertrophic chondrocyte ECM, that contains no living cells.

Negative Control 1. Oral Fibroblasts—MitC Treated+BMCs.

1×10⁶ oral fibroblasts were seeded in 3 petri dishes (10 cm), this is also 40% confluence level. 10 ml of growth medium was added and the cells are incubated at 33° C. and 5% CO₂, for 18 hours. After this time they were treated with 10 ul of mitomycin C in the same manner as described for the hypertrophic chondrocytes. Once treated the cells were washed with PBS, and 10 ml of bone juice was added as was 0.5 ml rat BMC suspension. The cells were incubated at 37° C. and 5% CO₂, and the medium changed at the same time as the other groups.

Oral fibroblasts were termed the negative control group as no increase in stimulation of BMC differentiation was expected, and to prove the results were cell specific.

Negative Control 2. Oral Fibroblasts—MitC Treated, Grown for 1 Week+BMCs.

1×10⁶ oral fibroblasts were seeded in 4 petri dishes (10 cm) and 10 ml of growth medium was added and the cells are incubated at 33° C. and 5% CO₂, for 18 hours. After this time they are treated with 10 ul of mitomycin-C (1 mg/ml). Once treated the cells were washed with PBS, and 10 ml growth medium was added to each dish and the cells were incubated at 33° C. and 5% CO₂ for a period of 1 week. The growth medium was changed twice that week.

After 1 week the growth medium was removed and the cells were washed with PBS. 10 ml bone juice was added to 5×10⁵ rat BMCs and the resulting suspension added to each of the petri dishes. They were incubated at 37° C. and 5% CO₂. The medium changed as specified.

Other Control Groups. Hypertrophic Chondrocytes— MitC Treated, Freeze-Thawed.

6 petri dishes were plated with 1.4×10⁶ cells, and incubated in 10 ml growth medium at 33° C. and 5% CO₂ for 18 hours. The next day, 3 of these dishes were freeze—thawed, and 3 mitomycin-C treated and to all of them 10 ml bone juice was added. These controls groups were treated exactly as the other groups however no rat BMCs were added. 3 petri dishes with mitomycin C treated oral fibroblasts were also seeded, and cultured in bone juice with no BMCs added.

Key Microscopic Observations

Day 6: Large ‘milky’ colonies were visible by eye, in the plates for treatment group 2 (Hypertrophic Chondrocytes—MitC treated, incubated for 1 week, freeze-thawed+BMCs). This was consistent with the appearance of calcified nodules.

Day 10. Milky colonies beginning to appear in the other treatment groups.

The experiment was stopped after 11 full days, and all the dishes were fixed and stained for the different colonies as described in the materials and methods section. The petri dishes were photographed and one photograph from each treatment and control group, for the different stains (CFU-AP, CFU-Ca, CFU-Col, and CFU-f), are shown in the following figures.

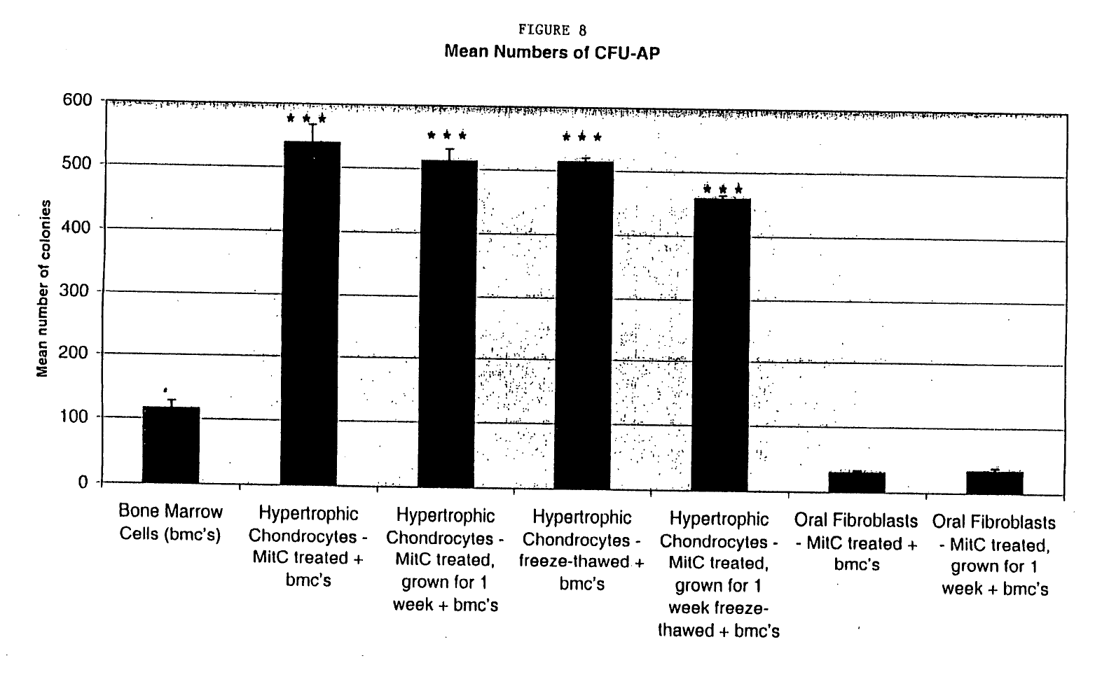

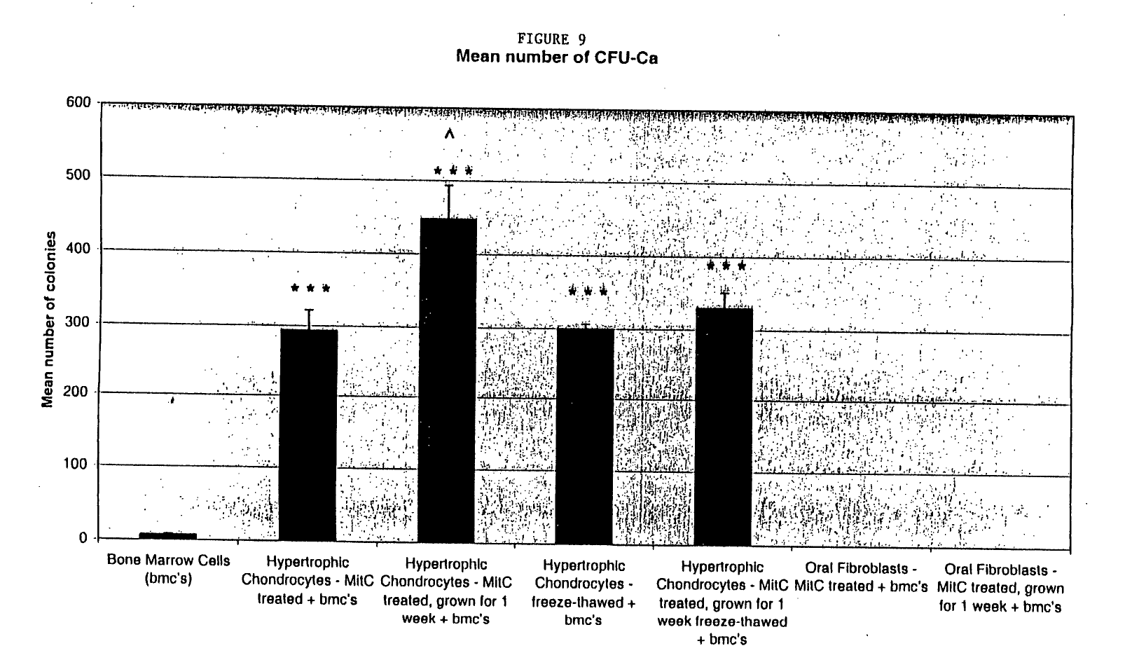

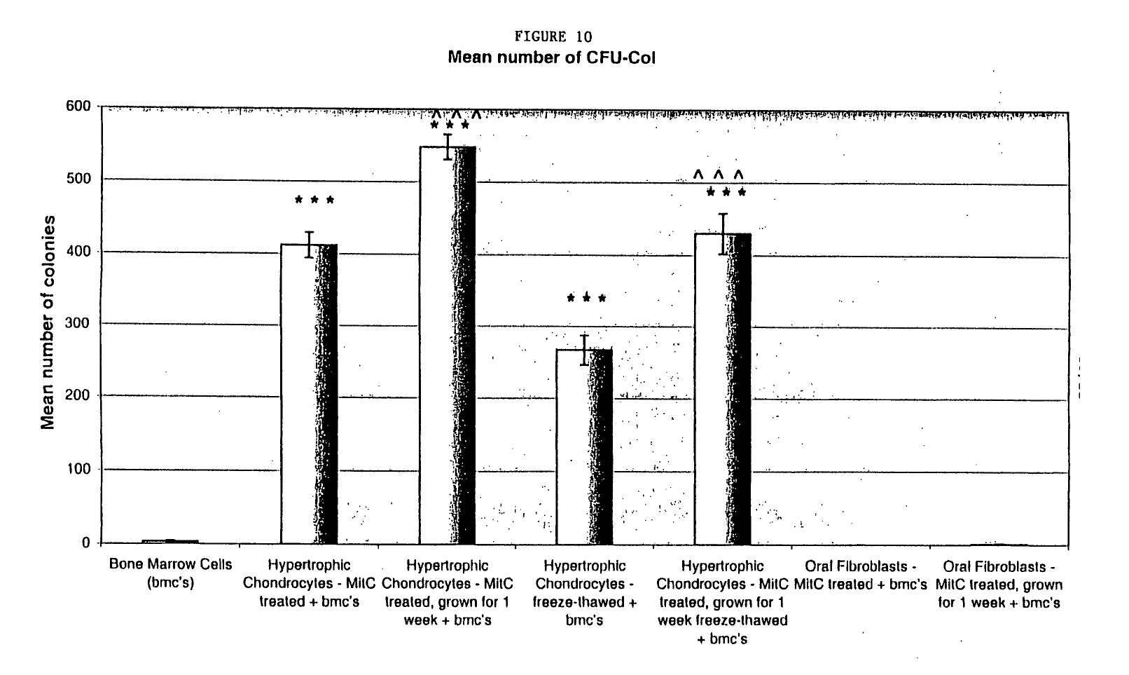

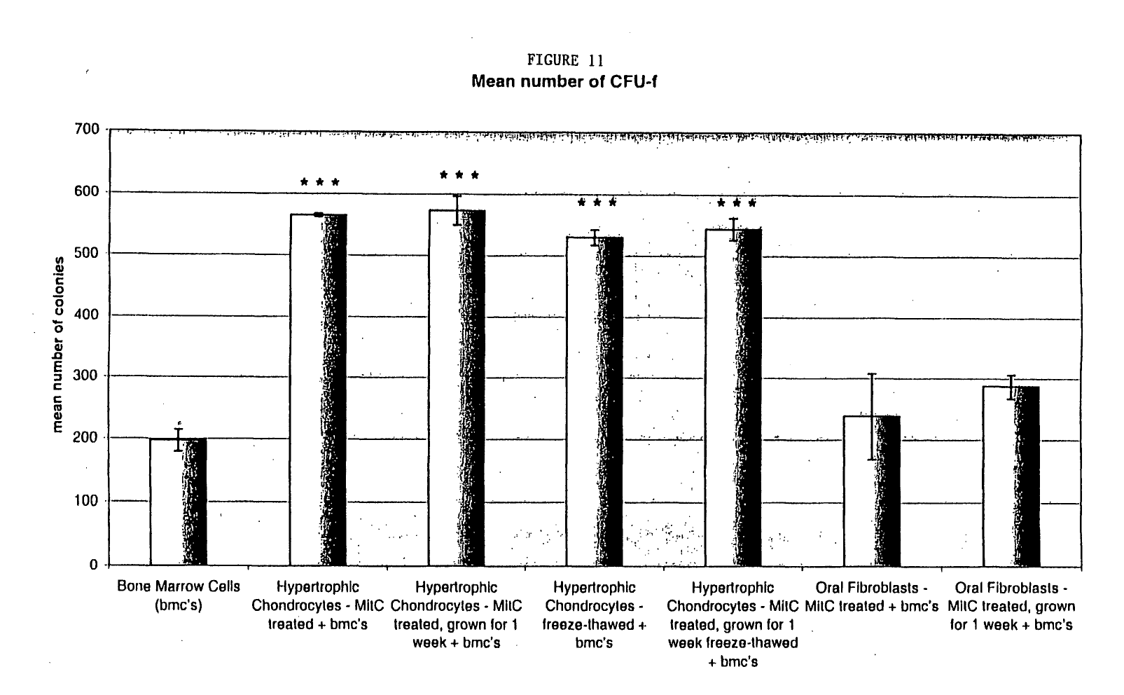

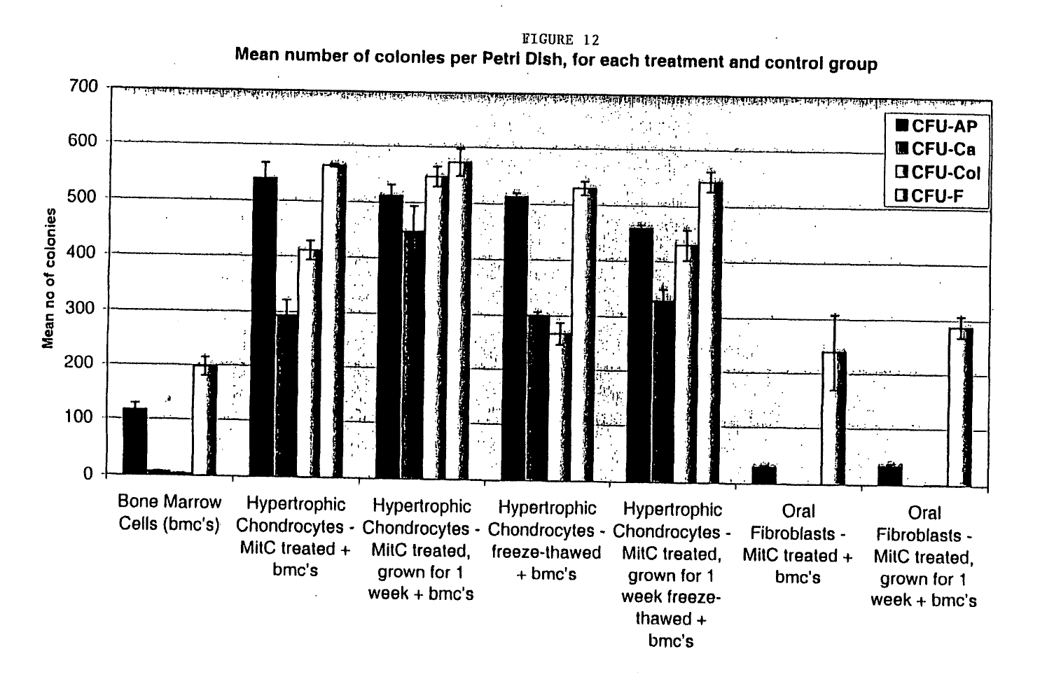

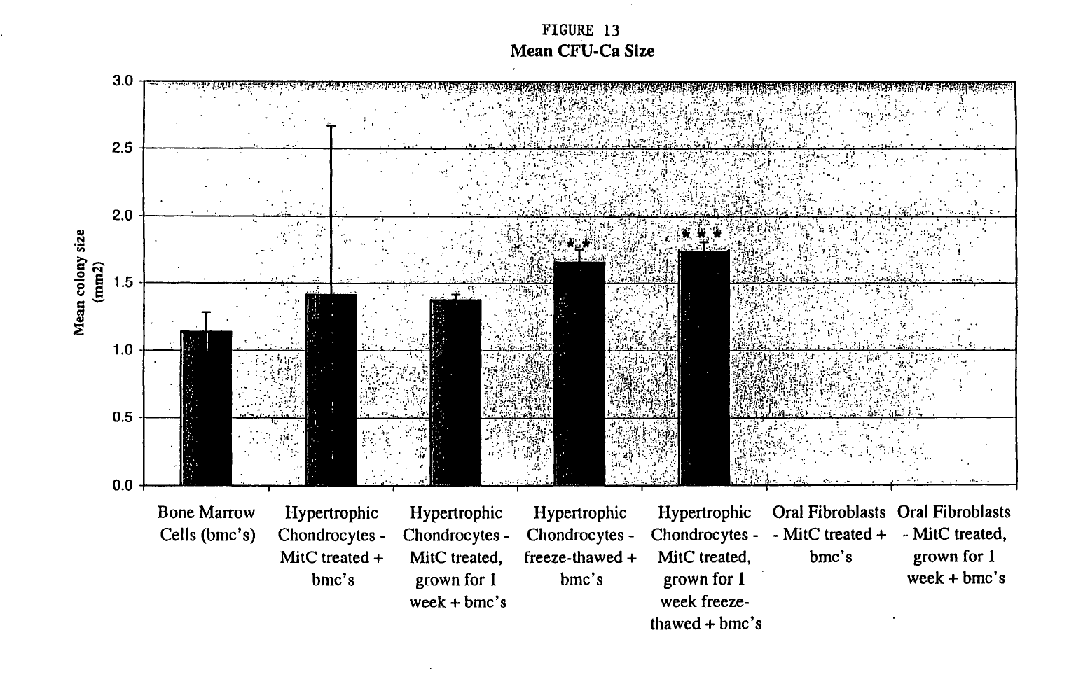

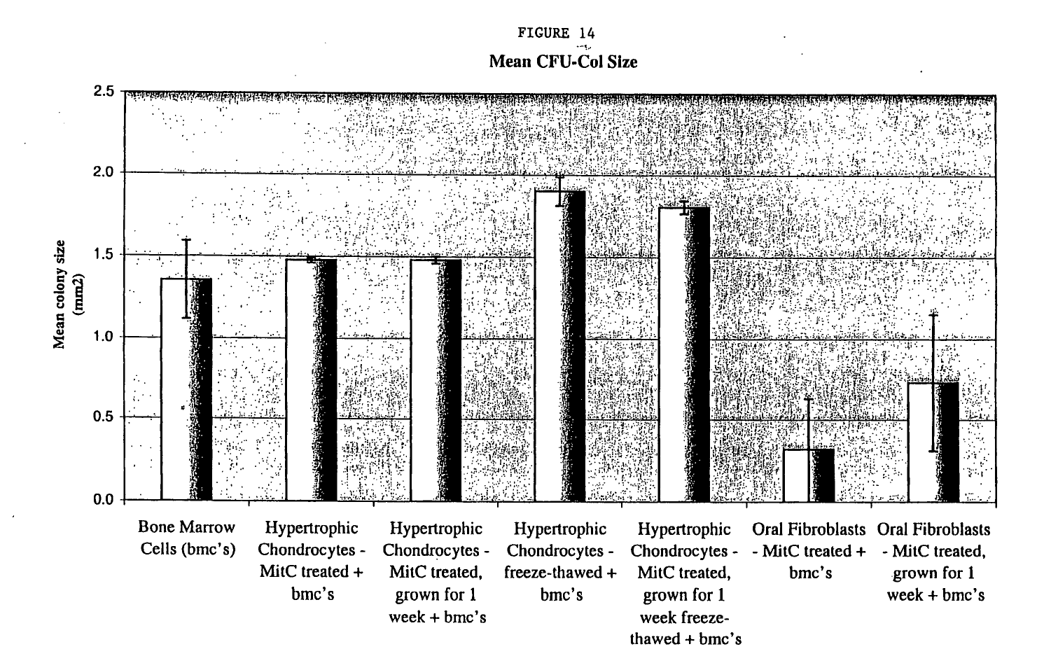

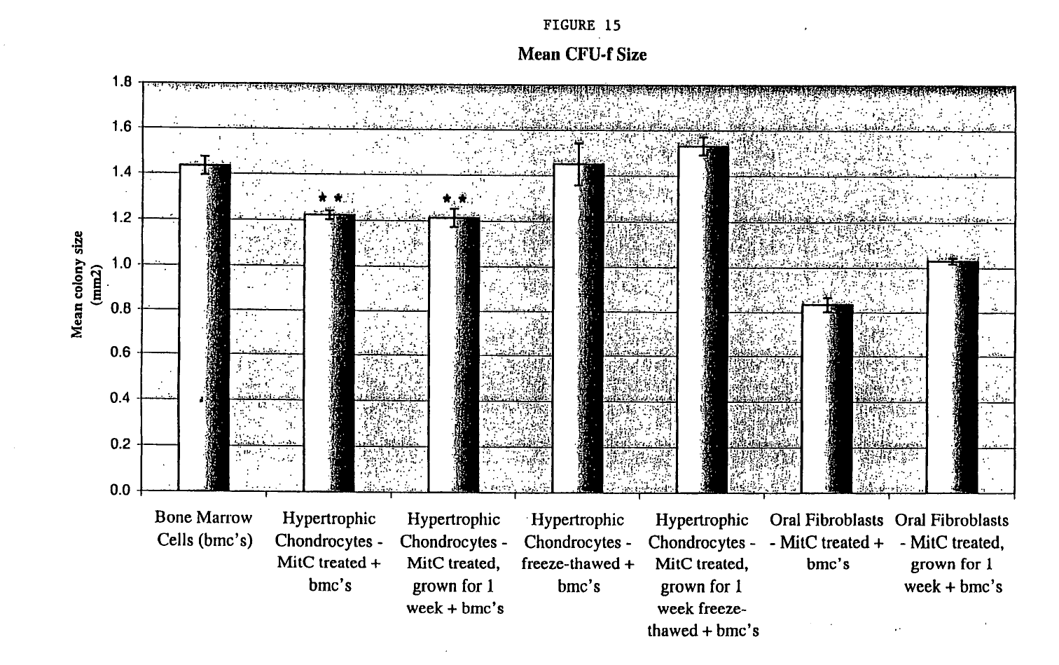

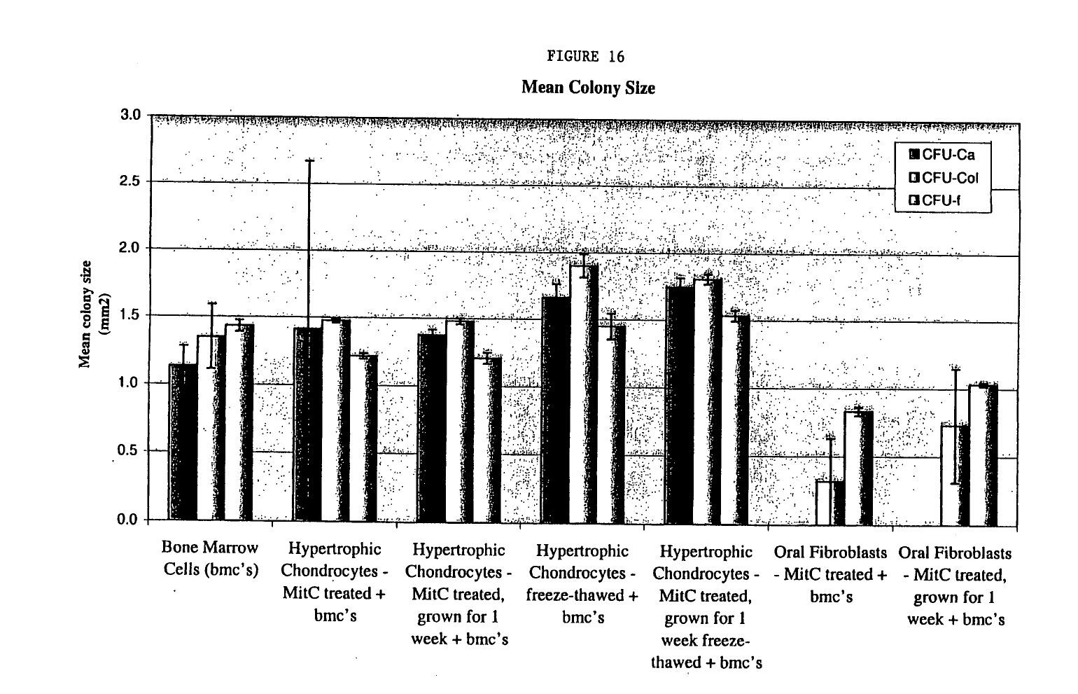

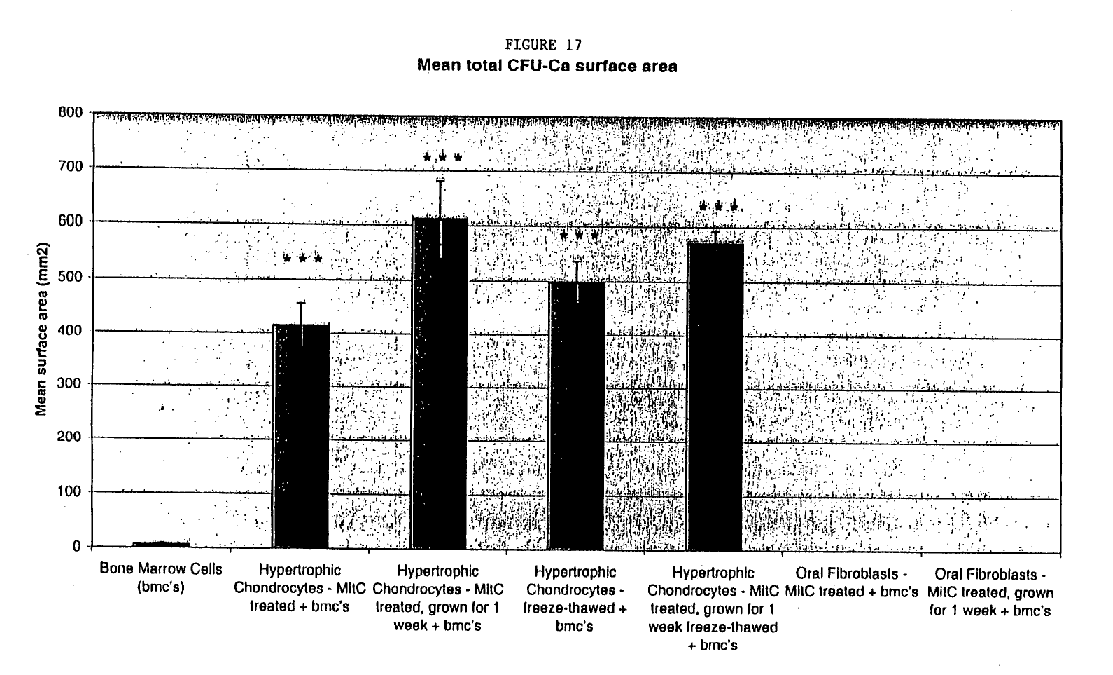

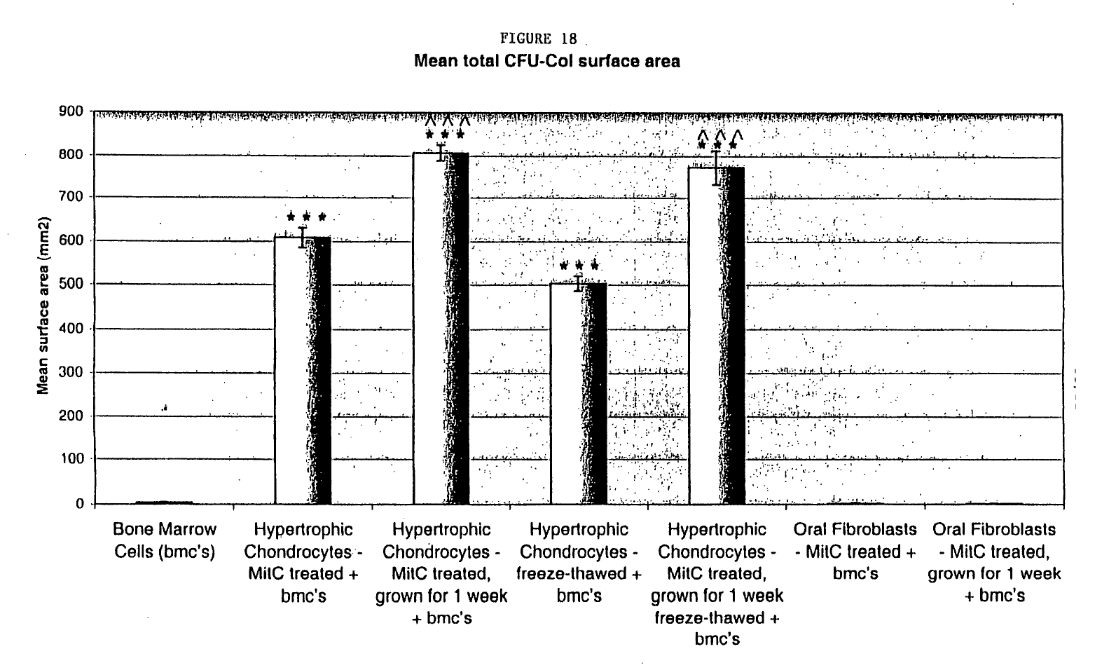

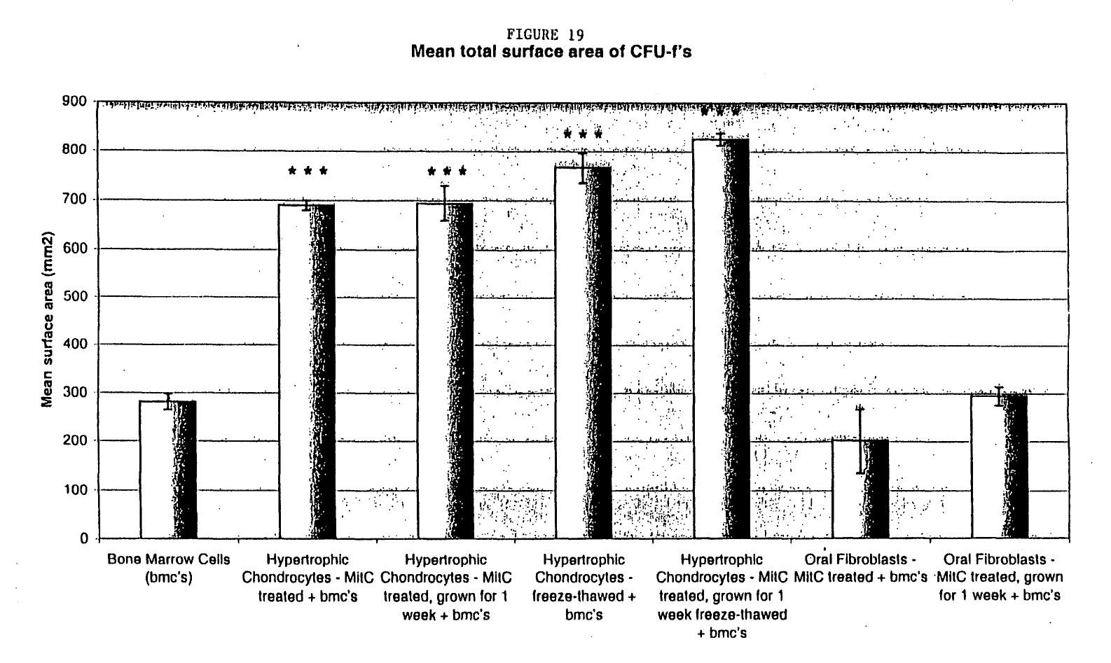

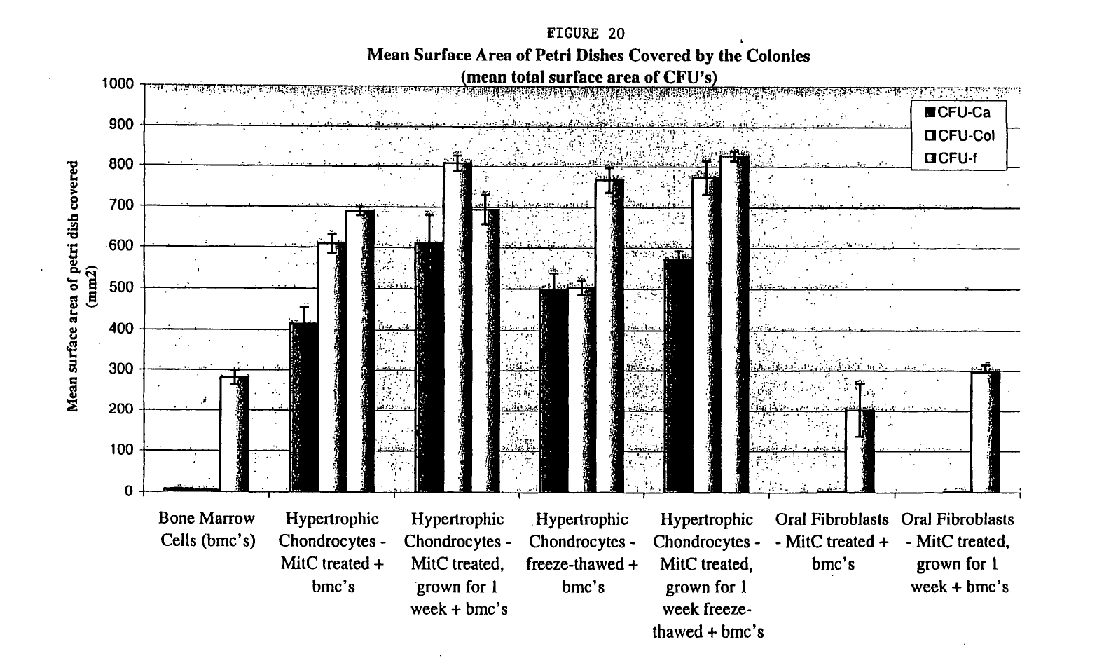

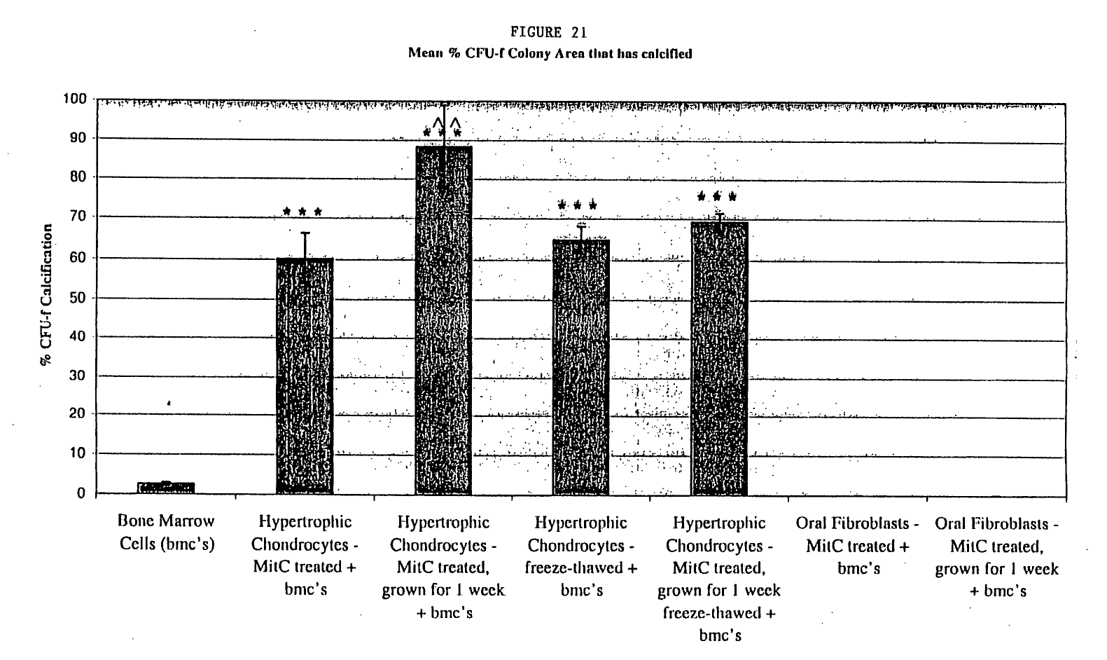

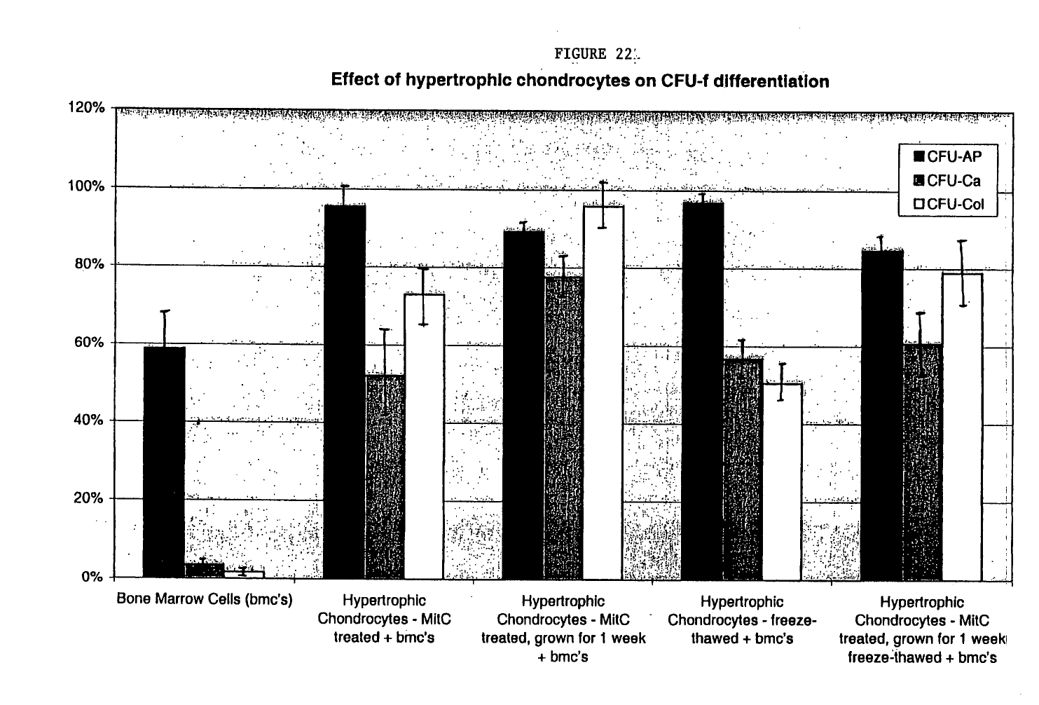

Key Observations After Quantitative Analysis as Described Through Tables 1 Through 19, and Charts 1 Through 15. (FIGS. 8 Through 22).

Chart 1. Shows the number of Alkaline positive colonies (CFU-AP) per is treatment and control group, as the mean number of colonies per petri dish (n=3,4)+SEM.

ANOVA

Significant differences between mean values of the treatment groups compared to the control, p<0.001.

Fisher LSD Multiple Comparison Test (a=0.05)

* * * Significant difference between treatment group and untreated control group 1 (p<0.001).

No significant differences between corresponding treatment groups (1 vs 2, 3 vs 4).

Chart 2. Shows the number of calcium positive colonies (CFU-Ca) per treatment and control group, as the mean number of colonies per petri dish (n=3,4)±SEM.

ANOVA

Significant differences between mean values of the treatment groups compared to the control, P<0.001.

Fisher LSD Multiple Comparison Test (a=0.05)

* * * Significant difference (p<0.001) between treatment group and untreated control group 1.

\hat{ } Significant difference (p<0.05) between corresponding treatment groups (1 vs 2).

Chart 3. Shows the number of collagen staining colonies (CFU-Col) per treatment and control group, as the mean number of colonies per petri dish (n=3,4)±SEM.

ANOVA

Significant differences between mean values of the treatment groups compared to the control, p<0.001.

Fisher LSD Multiple Comparisons Test (a=0.05)

* * * Significant difference (p<0.001) between treatment group and untreated control group 1.

\hat{ } \hat{ } \hat{ } Significant difference (p<0.001) between corresponding treatment groups (1 vs 2,3 vs 4).

Chart 4. Shows the number of CFU-fs per treatment and control group, as the mean number of colonies per petri dish (n=3,4)±SEM.

ANOVA

Significant differences between mean values of the treatment groups compared to the control, p<0.001.

Fisher LSD Multiple Comparison Test (a=0.05)

* * * Significant difference (p<0.001) between treatment group and untreated control group 1.

No significant differences between corresponding treatment groups (1 vs 2, 3 vs 4).

Chart 5. Shows the number of colonies (CFU-AP, CFU-Ca, CFU-Col, CFU-f) per treatment and control group, as the mean number of colonies per petri dish (n=3,4)±SEM.

Chart 6. Shows the mean size of calcium positive colonies (CPU-Ca) per treatment and control group, as the mean size (mm²) of colonies per petri dish is (n=3,4)±SEM.

ANOVA

Significant differences between mean values of the treatment groups compared to the control, p=0.003.

Fisher LSD multiple Comparison Test (a=0.05)

* * Significant difference (p<0.01) between treatment group and control group 1.

* * * Significant difference (p<0.001) between treatment group and untreated control group 1.

No significant differences between corresponding treatment groups (1 vs 2, 3 vs 4).

Chart 7. Shows the mean size of collagen staining colonies (CFU-Col) per treatment and control group, as the mean size (mm²) of colonies per petri dish (n=3,4)±SEM.

ANOVA

No significant differences between mean values of the treatment groups compared to the control.

Chart 8. Shows the mean size of CFU-fs per treatment and control group, as the mean size (mm²) of colonies per petri dish (n=3,4)±SEM.

ANOVA

Significant differences between mean values of the treatment groups compared to the control, p<0.001.

Fisher LSD Multiple Comparison Test (s=0.05)

* * Significant difference (p<0.01) between treatment group and control group 1.

No significant differences between corresponding treatment groups (1 vs 2, 3 vs 4).

Chart 9. Shows the mean size of colonies (CFU-Ca, CFU-Col, CFU-f) per treatment and control group, as the mean size (mm²) of colonies per petri dish (n=3,4)±SEM.

Chart 10. Shows the mean total surface area covered by the calcium positive colonies (CFU-Ca) per treatment and control group, as the mean total surface area (mm²) of colonies per petri dish (n=3,4)±SEM.

ANOVA

Data was transformed as In(x), to give more equal variances. Significant differences between mean values of the treatment groups compared to the control, p<0.001.

Fisher LSD Multiple Comparison Test (a=0.05)

* * * Significant difference (p<0.001) between treatment group and untreated control group 1.

No significant differences between corresponding treatment groups (1 vs 2, 3 vs 4).

Chart 11. Shows the mean total surface area covered by the collagen positive colonies (CFU-Col) per treatment and control group, as the mean total surface area (mm²) of colonies per petri dish (n=3,4)±SEM.

ANOVA

Significant differences between mean values of the treatment groups compared to the control, p<0.001).

Fisher LSD Multiple Comparison Test (a=0.05)

* * * Significant difference (p<0.001) between treatment group and untreated control group 1.

\hat{ } \hat{ } \hat{ } Significant difference (p<0.001) between corresponding treatment groups (1 vs 2, 3 vs 4).

Chart 12. Shows the mean total surface area covered by CFU-fs per treatment and control group, as the mean total surface area (mm²) of colonies per petri dish (n=3,4)±SEM.

ANOVA

Significant differences between mean values of the treatment groups compared to the control, p<0.001.

Fisher LSD Multiple Comparison Test (a=0.05)

* * * Significant differences (p<0.001) between treatment group and untreated control group 1.

No significant differences between corresponding treatment groups (1 vs 2, 3 vs 4).

Chart 13. Shows the mean total surface area covered by the colonies (CFU-Ca, CFU-Col, CFU-fs) per treatment and control group, as the mean total surface area (mm²) of colonies per petri dish (n=3,4)±SEM.

Chart 14. By using the data for total CFU-Ca surface area and CFU-f surface area we were able to calculate the % CFU-f surface that has calcified per petri dish. This chart shows the mean % surface area of CFU-fs that has calcified per treatment and control group, as the mean % total surface area (mm²) of CFU-Fs that has calcified per petri dish (n=3,4)±SEM.

ANOVA

Significant differences between mean values of the treatment groups compared to the control, p<0.001.

Fisher LSD Multiple Comparison Test (a=0.05)

* * * Significant difference (p<0.001) between treatment group and untreated control group 1.

\hat{ } \hat{ } A A Significant difference (p<0.01) between corresponding treatment groups (1 vs 2).

Chart 15. The Percentage CFU-f Differentiation

Using the data in Table 20, the percentage CFU-f colonies, that stained positive for alkaline phosphatase (CFU-AP), calcium (CFU-Ca), and collagen (CFU-Col), where calculated. This represents the percentage CFU-differentiation.

Results Tables

The results were analysed using MS Excel and Sigma Stat, and are presented in Tables 1 to 19. Three different groups of data were extracted; the mean number of colonies, the mean colony size (mm²) and the mean total surface area (mm²) covered by the colonies. The data is presented as the value per petri dish, standard deviation of results, mean value per treatment and control group, and the standard error of the mean (SEM).

However, due to the large number of CFU-APs, the Biolmage intelligent quantifier was unable to count these colonies hence they were counted manually, and thus the colony sizes and surface area data is not available.

By comparing the total surface area of the CFU-fs, to the total surface area of the CFU Ca, it was possible to determine the percentage of CFU-f area that had calcified.

Results Demonstrate That:

No effect found with oral fibroblasts

Immortalised human hypertrophic chondrocytes induce CFU-f formation, differentiation, and calcification of marrow cells—fundamental to osseoinductive activity.

The ossified nodules (Cfu-fs) express alkaline phosphatase activity, and elaborate an extracellular matrix which mineralises to form calcified nodules which have similar ultrastructural properties to calcified bone.

Surprisingly, the affects occur in the absence of living hypertrophic chondrocytes demonstrating that living cells are not required to induce marrow stromal cell to differentiate into mineralising osteoblasts.

### EXAMPLE 2

While grown in culture at either 33° C., and surprisingly at the permissive temperature of the active oncogene (37-39° C.), the immortalised human hypertrophic chondrocyte-like cells (HHC) expressed material which is released into the cell medium as, seemingly, cell debris or secreted extracellular matrix by the HHCs. The extracellular material produced is not restricted to a single clone but is a general characteristic of the skeletal cell lines that have been produced. A study was performed to see whether the material produced by these cells was capable of inducing the differentiation of marrow stromal cells into mineralising osteoblasts capable of elaborating a matrix, and secreting osteoblast marker proteins.

HHC cell “matrix” was collected as described in the materials and methods above and added to flasks containing bone marrow cells derived from rat femurs, or from human bone marrow biopsy material. In all cases the matrix—which was harvested from HHC medium, pelleted by centrifugation, separated from the overlying supernatant, and used either immediately or frozen before use—was able to induce human and/or rat marrow stromal cell differentiation. The marrow cells form CFU-Fs which elaborate and mineralise an extracellular matrix. Furthermore, the marrow cells express increasing amounts of osteocalcin (which is a marker for differentiated osteoblasts) in response to increasing concentrations of the matrix added.

### EXAMPLE 3

Electrophoretic Analysis of the Bioactive Matrix Material Harvested from Immortalised Human Hypertrophic Cartilage Cells

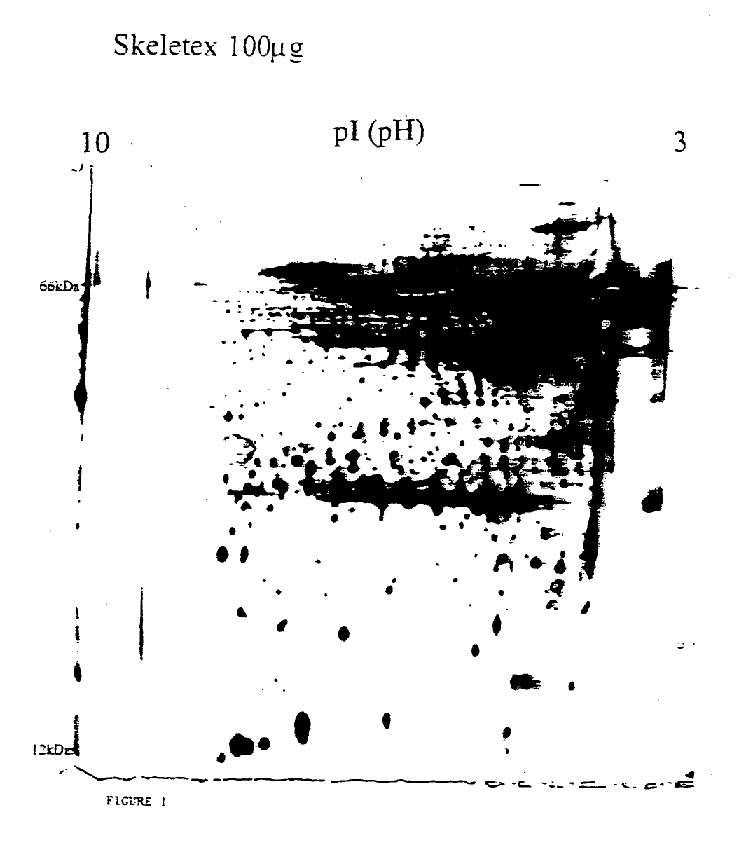

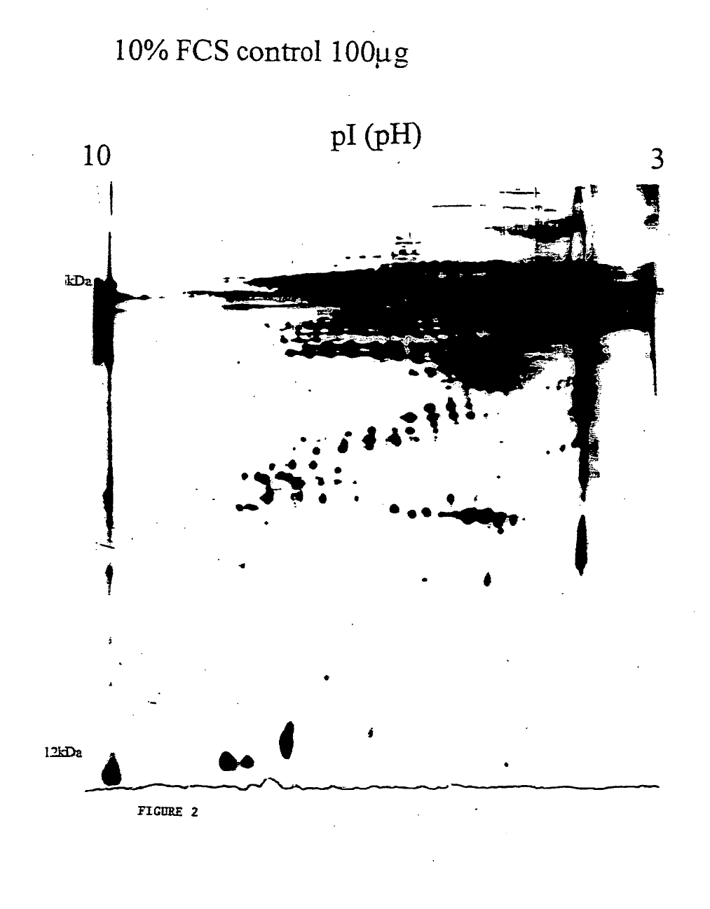

In order to determine the components of the HHC harvested material polyacrylamide gel electrophoresis was performed, and one and two dimensional gel analysis used to isolate the proteins present. Examples of the 2-D gel analysis are provided in FIGS. 1 and 2.

The results demonstrate that the extracellular matrix harvested from the HHC cells comprises a complex mix of noncollagenous and collagenous matrix proteins some of which are glycosylated, and, in addition, numerous cytokines and growth factors. It shows clearly a very complex mix comprised of potentially hundreds of proteins of various sizes and mobilities.

As described in the introductory sections, the natural processes of endochondral and intramembranous ossification require a complex myriad of signalling, over a sustained period of time, to be completed. This is performed by numerous factors, many unknown, which act in concert, and in a temporal fashion, to play out the symphony of bone (re)generation. It is not surprising, therefore, that the matrix produced by HHC cells, which has osseoinductive activity, comprise numerous proteins. Also, it stands to reason that loss of activity of even a single factor involved in the process of new bone formation could effect the outcome of skeletal repair and regeneration.

### EXAMPLE 4

Effects of Freeze-Dried HHC Matrix on Marrow Stromal Cells In Vitro.

HHC matrix was harvested and freeze-dried (a process known to have a detremental affect on the biological activity of numerous proteins) as described in the above materials and methods, and incubated with marrow stromal cells derived either from rat femurs as outlined above, or from human marrow biopsy material.

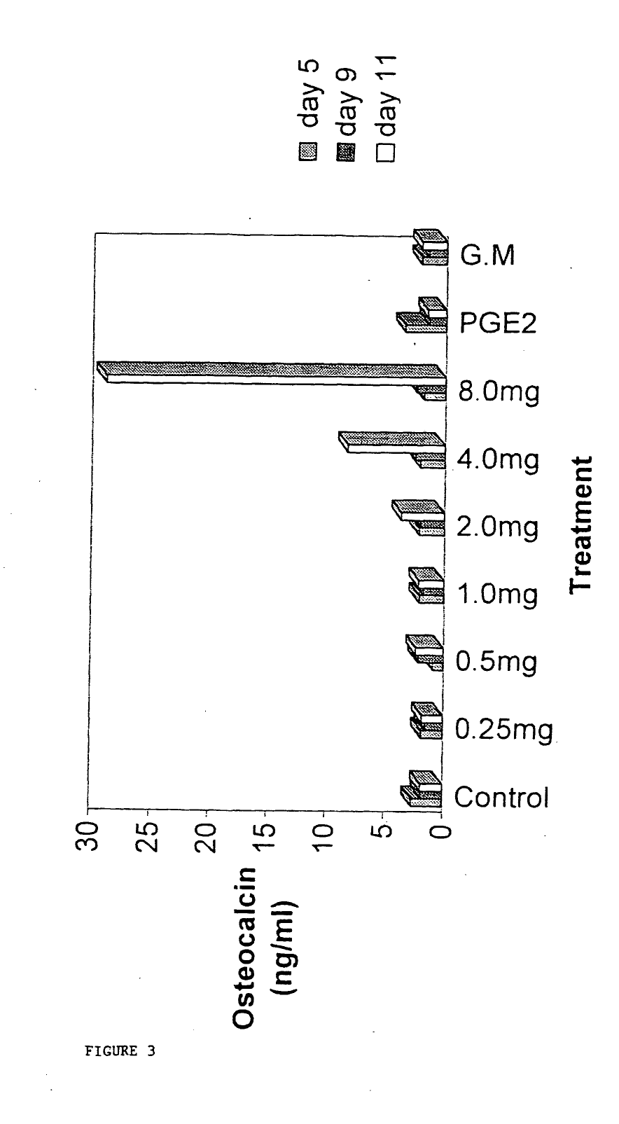

Surprisingly, the freeze-dried HHC matrix, despite comprising a complex mix of proteins which would be expected to lose aspects of biological activity, still maintains osseoinductive activity. Furthermore, the activity increases in a dose dependent manner as shown in FIG. 3. FIG. 3 shows increasing expression of osteocalcin by rat marrow cells incubated with various concentrations of freeze-dried HHC matrix up to 8 mg. The HHC matrix is added to medium on day 1 and day 5, days 1, 5, and 9, and days 1, 5, 9, and 11. Controls are marrow cells alone, or marrow cells plus prostaglandin E2 which is known to induce CFU-f formation in marrow cultures.

### EXAMPLE 5

Effects of Freeze-Dried HHC Matrix on Marrow Stromal Cells In Vivo.

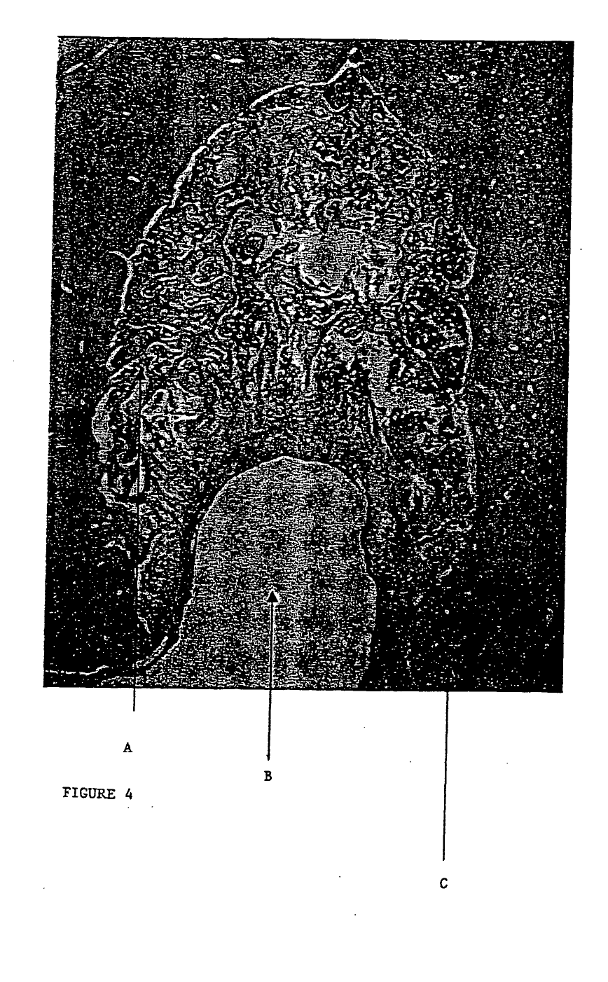

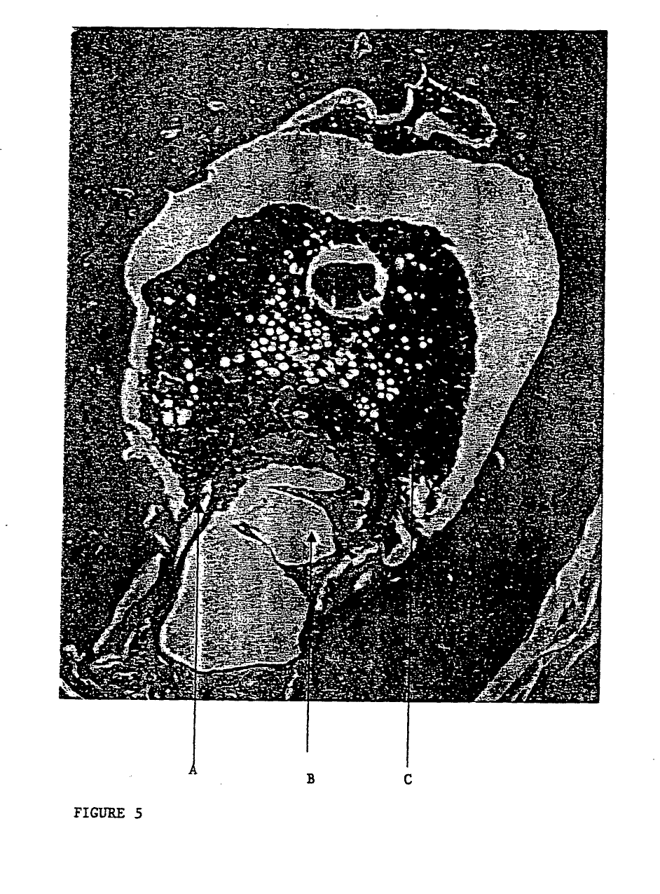

An initial controlled pilot study has examined the ability of freeze-dried HHC matrix to induce the formation of new bone in vivo. A series of young adult rats were operated and freeze-dried HHC matrix placed in holes made through the cortical bone of femurs to expose the marrow cavity. Placement of the freeze-dried matrix into the holes, which were then sealed with dental wax, resulted in significant new bone formation that is supported by a profoundly vascular stroma. It was concluded that the extent of the new bone formation far exceeds that which could be attributed to healing of the surgical effects. FIGS. 4 and 5 show control and treated histological samples from the study, respectively.

This was a wholly surprising result for two reasons. First, that freeze-dried matrix derived from a human cell line would have the signalling ability to induce, and complete, endochondral bone formation to produce new, highly vascularised bone in vivo was astonishing. Second, that this response was seen in the absence of expected untoward inflammatory effects due to cross-species (human material to non-immunosuppressed rodents) grafting.

### EXAMPLE 6

Demonstration That Low Levels of Bioactive Factors Exist in the Osseoinductive HHC Matrix.

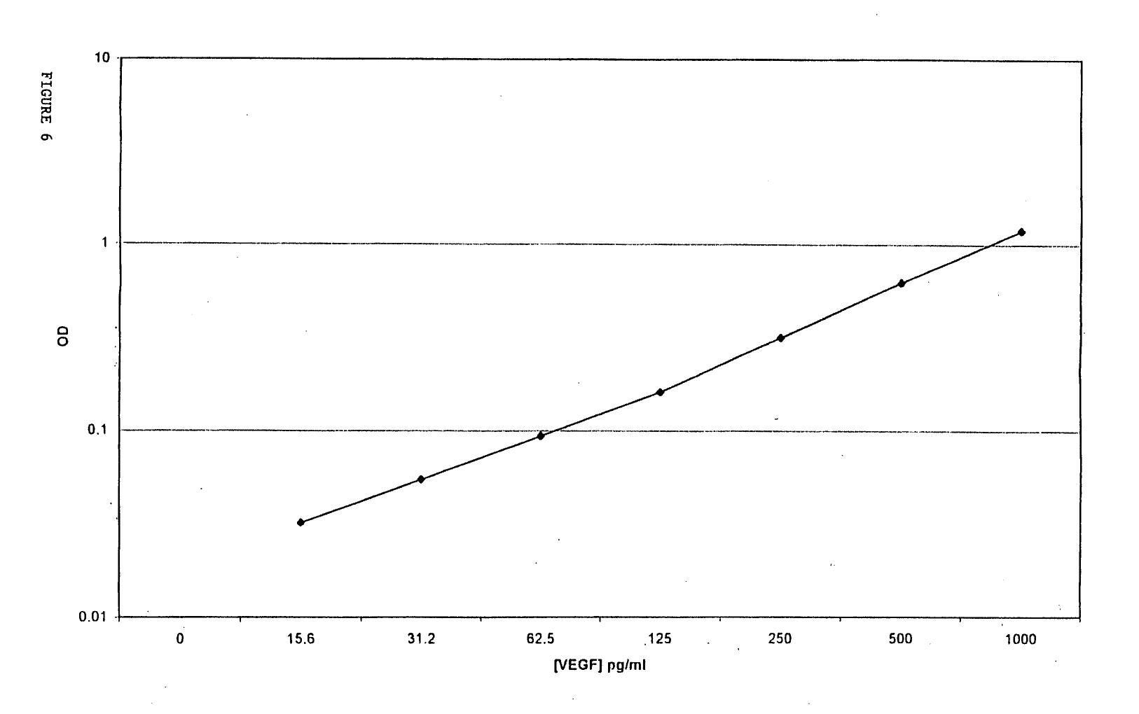

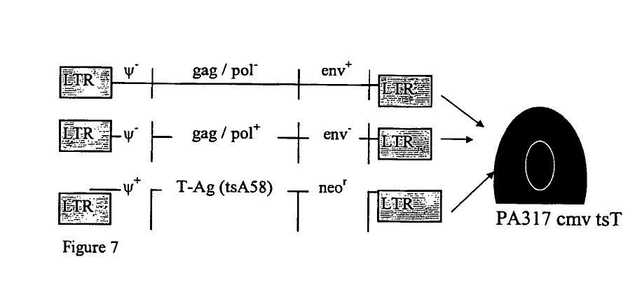

A study was performed to identify the amounts of biologically active factors that could be found in the HHC matrix. An assay of vascular endothelial growth factor, VEGF, (commercially available tes), was used to demonstrate that only picogramme quantities (up to 500 pg) of such growth factors were present per milligramme of freeze-dried matrix. Since it is known that as much is as 30 mg of pure BMP growth factor is required to induce a limited bone forming response in vivo, it was surprising that such a small amount of growth factor was enough to induce a full osseoinductive response in the in vivo study—generating highly vascular trabecular bone. Table 20 shows the picogramme levels of VEGF present per milligramme of freeze-dried HHC matrix; as determined from the VEGF standard curve generated (FIG. 6).

TABLE 1
Number of colonies per petri dish.

| | Number of colonies per petri dish | | | |
|---|---|---|---|---|
| | CFU-AP | CFU-Ca | CFU-Col | CFU-f |
| Bone Marrow Cells (BMC's) | 93 | 5 | 2 | 179 |
| | 133 | 6 | 6 | 187 |
| | 140 | 11 | 5 | 247 |
| | 97 | 6 | 1 | 176 |
| Hypertrophic Chondrocytes - | 589 | 334 | 418 | 569 |
| MitC treated + bmc's | | | | |
| | 491 | 238 | 379 | 559 |
| | 539 | 308 | 437 | 565 |
| Hypertrophic Chondrocytes - | 545 | 498 | 573 | 575 |
| MitC treated, grown for | | | | |
| 1 week + bmc's | | | | |
| | 501 | 355 | 561 | 544 |
| | 548 | 386 | 495 | 533 |
| | 542 | 550 | 560 | 639 |
| Hypertrophic Chondrocytes - | 524 | 291 | 272 | 548 |
| freeze-thawed + bmc's | | | | |
| | 513 | 295 | 299 | 535 |
| | 501 | 313 | 229 | 506 |
| Hypertrophic Chondrocytes - | 462 | 273 | 369 | 519 |
| MitC treated, grown for 1 | | | | |
| week freeze-thawed + bmc's | | | | |
| | 460 | 371 | 501 | 569 |
| | 444 | 333 | 413 | 507 |
| | 471 | 344 | 435 | 579 |
| Oral Fibroblasts - | 35 | 0 | 0 | 191 |
| MitC treated + bmc's | | | | |
| | 28 | 0 | 0 | 150 |
| | 24 | 0 | 0 | 375 |
| Oral Fibroblasts - MitC | 27 | 0 | 1 | 333 |
| treated, grown for | | | | |
| 1 week + bmc's | | | | |
| | 24 | 0 | 0 | 251 |
| | 37 | 0 | 3 | 258 |
| | 43 | 0 | 0 | 306 |
| Oral Fibroblasts - MitC | 0 | 0 | 0 | 0 |
| treated | | | | |
| | 0 | 0 | 0 | 0 |
| | 0 | 0 | 0 | 0 |
| Hypertrophic Chondrocytes - | 0 | 0 | 0 | 0 |
| MitC treated | | | | |
| | 0 | 0 | 0 | 0 |
| | 0 | 0 | 0 | 0 |
| Hypertrophic Chondrocytes - | 0 | 0 | 0 | 0 |
| freeze-thawed | | | | |
| | 0 | 0 | 0 | 0 |
| | 0 | 0 | 0 | 0 |

TABLE 2
Mean number of colonies per petri dish,
for each treatment and control group.

| | Mean number of colonies per dish, | | | |
|---|---|---|---|---|
| | for each treatment and control group | | | |
| | CFU-AP | CFU-Ca | CFU-Col | CFU-f |
| Bone Marrow Cells (bmc's) | 115.75 | 7.00 | 3.50 | 197.25 |
| Hypertrophic Chondrocytes - | 539.67 | 293.33 | 411.33 | 564.33 |
| MitC treated + bmc's | | | | |
| Hypertrophic Chondrocytes - | 511.50 | 447.25 | 547.25 | 572.75 |
| MitC treated, gorwn for | | | | |
| 1 week + bmc's | | | | |
| Hypertrophic Chondrocytes - | 512.67 | 299.67 | 266.57 | 529.57 |
| freeze-thawed + bmc's | | | | |
| Hypertrophic Chondrocytes - | 459.25 | 330.25 | 429.50 | 543.50 |
| MitC treated, grown for 1 | | | | |
| week freeze-thawed + bmc's | | | | |
| Oral Fibroblasts - | 29.00 | 0.00 | 0.00 | 238.67 |
| MitC treated + bmc's | | | | |
| Oral Fibroblasts - | 32.75 | 0.00 | 1.00 | 287.00 |
| freeze-thawed + bmc's | | | | |
| Oral Fibroblasts - | 0.00 | 0.00 | 0.00 | 0.00 |
| MitC treated, grown for | | | | |
| 1 week + bmc's | | | | |
| Oral Fibroblasts - | 0.00 | 0.00 | 0.00 | 0.00 |
| MitC treated, grown for 1 | | | | |
| week freeze-thawed + bmc's | | | | |

TABLE 3
Standard deviation of results for the number of colonies
per petri dish, for each treatment and control group.

| | Number of colonies per petri dish | | | |
|---|---|---|---|---|
| | Standard Deviation | | | |
| | CFU-AP | CFU-Ca | CFU-Col | CFU-f |
| Bone Marrow Cells (bmc's) | 24.19 | 2.71 | 2.38 | 33.49 |
| Hypertrophic Chondrocytes - | 49.00 | 49.55 | 29.57 | 5.03 |
| MitC treated + bmc's | | | | |
| Hypertrophic Chondrocytes - | 40.93 | 92.00 | 35.33 | 47.61 |
| MitC treated, gorwn for | | | | |
| 1 week + bmc's | | | | |
| Hypertrophic Chondrocytes - | 11.50 | 11.72 | 35.30 | 21.50 |
| freeze-thawed + bmc's | | | | |
| Hypertrophic Chondrocytes - | 11.24 | 41.37 | 55.00 | 35.79 |
| MitC treated, grown for 1 | | | | |
| week freeze-thawed + bmc's | | | | |
| Oral Fibroblasts - | 5.57 | 0.00 | 0.00 | 119.83 |
| MitC treated + bmc's | | | | |
| Oral Fibroblasts - | 8.81 | 0.00 | 1.41 | 39.22 |
| MitC treated, grown for | | | | |
| 1 week + bmc's | | | | |

TABLE 4
(SEM) Standard error of the mean number of colonies
per dish, for each treatment and control group.

| | Number of colonies per petri dish | | | |
|---|---|---|---|---|
| | Standard Error of the Mean | | | |
| | CFU-AP | CFU-Ca | CFU-Col | CFU-f |
| Bone Marrow Cells (bmc's) | 12.09 | 1.35 | 1.19 | 16.75 |
| Hypertrophic Chondrocytes - | 28.29 | 28.67 | 17.07 | 2.91 |
| MitC treated + bmc's | | | | |
| Hypertrophic Chondrocytes - | 20.46 | 46.00 | 17.67 | 23.81 |
| MitC treated, grown for | | | | |
| 1 week + bmc's | | | | |
| Hypertrophic Chondrocytes - | 6.64 | 6.77 | 20.38 | 12.41 |
| freeze-thawed + bmc's | | | | |
| Hypertrophic Chondrocytes - | 5.62 | 20.69 | 27.50 | 17.90 |
| MitC treated, grown for 1 | | | | |
| week freeze-thawed + bmc's | | | | |
| Oral Fibroblasts - | 3.21 | 0.00 | 0.00 | 69.19 |
| MitC treated + bmc's | | | | |
| Oral Fibroblasts - | 4.40 | 0.00 | 0.71 | 19.61 |
| MitC treated, gorwn for | | | | |
| 1 week + bmc's | | | | |
| Oral Fibroblasts - | 0.00 | 0.00 | 0.00 | 0.00 |
| MitC treated | | | | |
| Hypertrophic Chondrocytes - | 0.00 | 0.00 | 0.00 | 0.00 |
| MitC treated | | | | |
| Hypertrophic Chondrocytes - | 0.00 | 0.00 | 0.00 | 0.00 |
| freeze-thawed | | | | |

TABLE 5
Mean colony size (mm²) per petri dish

| | Mean colony size (mm²) | | |
|---|---|---|---|
| | per petri dish | | |
| | CFU-Ca | CFU-Col | CFU-f |
| Bone Marrow Cells (bmc's) | 0.83 | 0.98 | 1.49 |
| | 1.09 | 1.28 | 1.40 |
| | 1.09 | 1.14 | 1.34 |
| | 1.55 | 2.02 | 1.51 |
| Hypertrophic Chondrocytes - | 1.44 | 1.49 | 1.19 |
| MitC treated − bmc's | | | |
| | 1.44 | 1.49 | 1.22 |
| | 1.36 | 1.45 | 1.26 |
| Hypertrophic Chondrocytes - | 1.45 | 1.46 | 1.09 |
| MitC treated, grown for 1 week + bmc's | | | |
| | 1.30 | 1.50 | 1.25 |
| | 1.44 | 1.53 | 1.26 |
| | 1.32 | 1.43 | 1.25 |
| Hypertrophic Chondrocytes - | 1.47 | 1.85 | 1.34 |
| freeze-thawed + bmc's | | | |
| | 1.70 | 1.78 | 1.38 |
| | 1.81 | 2.07 | 1.63 |
| Hypertrophic Chondrocytes - | 1.90 | 1.79 | 1.60 |
| MitC treated, grown for 1 week | | | |
| freeze-thawed + bmc's | | | |
| | 1.64 | 1.70 | 1.51 |
| | 1.81 | 1.87 | 1.59 |
| | 1.62 | 1.85 | 1.43 |
| Oral Fibroblasts - | 0.00 | 0.94 | 0.79 |
| MitC treated + bmc's | | | |
| | 0.00 | 0.00 | 0.83 |
| | 0.00 | 0.00 | 0.00 |
| Oral Fibroblasts - | 0.00 | 1.37 | 1.02 |
| MitC grown for | | | |
| 1 week + bmc's | | | |
| | 0.00 | 0.00 | 1.07 |
| | 0.00 | 1.54 | 1.01 |
| | 0.00 | 0.00 | 1.02 |
| Oral Fibroblasts - | 0.00 | 0.00 | 0.00 |
| MitC treated | | | |
| | 0.00 | 0.00 | 0.00 |
| | 0.00 | 0.00 | 0.00 |
| Hypertrophic Chondrocytes - | 0.00 | 0.00 | 0.00 |
| MitC treated | | | |
| | 0.00 | 0.00 | 0.00 |
| | 0.00 | 0.00 | 0.00 |
| Hypertrophic Chondrocytes - | 0.00 | 0.00 | 0.00 |
| freeze-thawed | | | |
| | 0.00 | 0.00 | 0.00 |
| | 0.00 | 0.00 | 0.00 |

TABLE 6
Mean colony size (mm²)
per treatment and control group

| | Mean colony size (mm²) | | |
|---|---|---|---|
| | per cell type and | | |
| | culture condition | | |
| | CFU-Ca | CFU-Col | CFU-f |
| Bone Marrow Cells (bmc's) | 1.14 | 1.35 | 1.43 |
| Hypertrophic Chondrocytes - | 1.41 | 1.48 | 1.22 |
| MitC treated + bmc's | | | |
| Hypertrophic Chondrocytes - | 1.66 | 1.90 | 1.45 |
| freeze-thawed + bmc's | | | |
| Hypertrophic Chondrocytes - | 1.38 | 1.48 | 1.21 |
| MitC treated, grown for 1 week + bmc's | | | |
| Hypertrophic Chondrocytes - | 1.74 | 1.80 | 1.53 |
| MitC treated, grown for 1 week | | | |
| freeze-thawed + bmc's | | | |
| Oral Fibroblasts - | 0.00 | 0.31 | 0.83 |
| MitC treated + bmc's | | | |
| Oral Fibroblasts - | 0.00 | 0.73 | 1.03 |
| MitC treated, grown for 1 week + bmc's | | | |

TABLE 7
Standard devistion of results for the mean colony size, in the
3-4 petri dishes for each treatment and control group.

| | Mean colony size (mm²) | | |
|---|---|---|---|
| | per petri dish | | |
| | CFU-Ca | CFU-Col | CFU-f |
| Bone Marrow Cells (bmc's) | 0.30 | 0.46 | 0.08 |
| Hypertrophic Chondrocytes - | 0.04 | 0.03 | 0.03 |
| MitC treated + bmc's | | | |
| Hypertrophic Chondrocytes - | 0.08 | 0.04 | 0.08 |
| MitC treated, grown for 1 week + bmc's | | | |
| Hypertrophic Chondrocytes - | 0.17 | 0.15 | 0.16 |
| freeze-thawed + bmc's | | | |
| Hypertrophic Chondrocytes - | 0.14 | 0.08 | 0.08 |
| MitC treated, grown for 1 week | | | |
| freeze-thawed + bmc's | | | |
| Oral Fibroblasts - | 0.00 | 0.55 | 0.05 |
| MitC treated + bmc's | | | |
| Oral Fibroblasts - | 0.00 | 0.84 | 0.03 |
| MitC treated, grown for 1 week + bmc's | | | |
| Oral Fibroblasts - | 0.00 | 0.00 | 0.00 |
| MitC treated | | | |
| Hypertrophic Chondrocytes - | 0.00 | 0.00 | 0.00 |
| MitC treated | | | |
| Hypertrophic Chondrocytes - | 0.00 | 0.00 | 0.00 |
| freeze-thawed | | | |

TABLE 8
(SEM) Standard error of the mean size of colonies
(mm²) for each treatment group.

| | Mean colony size (mm²) | | |
|---|---|---|---|
| | per petri dish | | |
| | Standard Error of the | | |
| | Mean (SEM) | | |
| | CFU-Ca | CFU-Col | CFU-f |
| Bone Marrow Cells (bmc's) | 0.15 | 0.24 | 0.04 |
| Hypertorphic Chondrocytes - | 1.25 | 0.01 | 0.02 |
| MitC treated + bmc's | | | |
| Hypertorphic Chondrocytes - | 0.04 | 0.02 | 0.04 |
| MitC treated, grown for 1 week + bmc's | | | |
| Hypertrophic Chondrocytes - | 0.10 | 0.09 | 0.09 |
| freeze-thawed + bmc's | | | |
| Hypertorphic Chondrocytes - | 0.07 | 0.04 | 0.04 |
| MitC treated, grown for | | | |
| 1 week freeze-thawed + bmc's | | | |
| Oral Fibroblasts - | 0.00 | 0.31 | 0.03 |
| MitC treated + bmc's | | | |
| Oral Fibroblasts - | 0.00 | 0.42 | 0.01 |
| MitC treated, grown for 1 week + bmc's | | | |
| Oral Fibroblasts - | 0.00 | 0.00 | 0.00 |
| MitC treated | | | |
| Hypertrophic Chondrocytes - | 0.00 | 0.00 | 0.00 |
| MitC treated | | | |
| Hypertrophic Chondrocytes - | 0.00 | 0.00 | 0.00 |
| freeze-thawed | | | |

TABLE 9
Mean total surface area (mm²) of the colonies per petri dish

| | Total surface area of colonies | | |
|---|---|---|---|
| | per petri dish (mm²) | | |
| | CFU-Ca | CFU-Col | CFU-f |
| Bone Marrow Cells (bmc's) | 4.16 | 1.96 | 266.8 |
| | 6.55 | 7.65 | 261.29 |
| | 9.28 | 5.69 | 330.84 |
| | 9.28 | 2.02 | 265.8 |
| Hypertrophic Chondrocytes - | 482.2 | 624.41 | 675.56 |
| MitC treated + bmc's | | | |
| | 341.72 | 563.54 | 684.16 |
| | 420.08 | 637.13 | 709.81 |
| Hypertrophic Chondrocytes - | 723.3 | 823.14 | 626.46 |
| MitC treated, grown for 1 | | | |
| week + bmc's | | | |
| | 436.31 | 848.59 | 678.03 |
| | 555.12 | 759.42 | 676.03 |
| | 726.04 | 799.03 | 795.14 |
| Hypertrophic Chondrocytes - | 427.33 | 502.45 | 732.07 |
| freeze-thawed + bmc's | | | |
| | 501.22 | 532.41 | 739.31 |
| | 565.44 | 473.72 | 826.73 |
| Hypertrophic Chondrocytes - | 519.83 | 659.89 | 811.52 |
| MitC treated, grown for 1 | | | |
| week freeze-thawed + bmc's | | | |
| | 606.72 | 852.67 | 861.42 |
| | 603.93 | 774.08 | 806.08 |
| | 556.79 | 803.56 | 827.53 |
| Oral Fibroblasts - | 0 | 4.72 | 148.66 |
| MitC treated + bmc's | | | |
| | 0 | 0 | 125.11 |
| | 0 | 0 | 332.47 |
| Oral Fibroblasts - | 0 | 1.37 | 340.63 |
| MitC treated, grown for 1 week + bmc's | | | |
| | 0 | 0 | 268.42 |
| | 0 | 4.63 | 259.78 |
| | 0 | 0 | 312.31 |
| Oral Fibroblasts - | 0 | 0 | 0 |
| MitC treated | | | |
| | 0 | 0 | 0 |
| | 0 | 0 | 0 |
| Hypertrophic Chondrocytes - | 0 | 0 | 0 |
| MitC treated | | | |
| | 0 | 0 | 0 |
| | 0 | 0 | 0 |
| Hypertrophic Chondrocytes - | 0 | 0 | 0 |
| freeze-thawed | | | |
| | 0 | 0 | 0 |
| | 0 | 0 | 0 |

TABLE 10
The table shows the mean total surface area (mm²)
of the colonies, for each of the
different treatment and control groups.

| | Mean total surface area | | |
|---|---|---|---|
| | of colonies (mm²) | | |
| | CFU-Ca | CFU-Col | CFU-f |
| Bone Marrow Cells (bmc's) | 7.32 | 4.33 | 281.18 |
| Hypertorphic Chondrocytes - | 414.67 | 608.36 | 689.84 |
| MitC treated + bmc's | | | |
| Hypertorphic Chondrocytes - | 610.19 | 807.54 | 693.91 |
| MitC treated, grown for 1 week + bmc's | | | |
| Hypertrophic Chondrocytes - | 498.00 | 502.86 | 766.04 |
| freeze-thawed + bmc's | | | |
| Hypertorphic Chondrocytes - | 571.82 | 772.55 | 826.64 |
| MitC treated, grown for | | | |
| 1 week freeze-thawed + bmc's | | | |
| Oral Fibroblasts - | 0.00 | 1.57 | 202.08 |
| MiC treated + bmc's | | | |
| Oral Fibroblasts - | 0.00 | 1.50 | 295.29 |
| MitC treated, grown for 1 week + bmc's | | | |

TABLE 11
Standard deviation of the total colony surface area,
in the sets of 3-4 petri dishes for each
treatment and control group.

| | Total surface area | | |
|---|---|---|---|
| | of the colonies | | |
| | Standard Deviation | | |
| | CFU-Ca | CFU-Col | CFU-f |
| Bone Marrow Cells (bmc's) | 2.47 | 2.82 | 33.19 |
| Hypertorphic Chondrocytes - | 70.40 | 39.33 | 17.82 |
| MitC treated + bmc's | | | |
| Hypertorphic Chondrocytes - | 140.81 | 37.93 | 71.57 |
| MitC treated, grown for 1 week + bmc's | | | |
| Hypertrophic Chondrocytes - | 69.11 | 29.35 | 52.69 |
| freeze-thawed + bmc's | | | |
| Hypertorphic Chondrocytes - | 41.54 | 81.80 | 24.91 |
| MitC treated, grown for | | | |
| 1 week freeze-thawed + bmc's | | | |
| Oral Fibroblasts - | 0.00 | 2.73 | 113.53 |
| MitC treated + bmc's | | | |
| Oral Fibroblasts - | 0.00 | 2.18 | 37.98 |
| MitC treated, grown for 1 week + bmc's | | | |
| Oral Fibroblasts - MitC treated | 0.00 | 0.00 | 0.00 |
| Hypertrophic Chondrocytes - | 0.00 | 0.00 | 0.00 |
| MitC treated | | | |
| Hypertrophic Chondrocytes - | 0.00 | 0.00 | 0.00 |
| freeze-thawed | | | |

TABLE 12
SEM (Standard error of the mean total surface area
(mm²) of the colonies, for each treatment
and control group.

| | Total surface area | | |
|---|---|---|---|
| | of the colonies | | |
| | Standard Error | | |
| | of the Mean (SEM) | | |
| | CFU-Ca | CFU-Col | CFU-f |
| Bone Marrow Cells (bmc's) | 1.23 | 1.41 | 16.60 |
| Hypertorphic Chondrocytes - | 40.64 | 22.71 | 10.29 |
| MitC treated + bmc's | | | |
| Hypertorphic Chondrocytes - | 20.77 | 40.90 | 12.46 |
| MitC treated, grown for | | | |
| 1 week freeze-thawed + bmc's | | | |
| Hypertrophic Chondrocytes - | 70.40 | 18.97 | 35.79 |
| MitC treated, grown for 1 week + bmc's | | | |
| Hypertorphic Chondrocytes - | 39.90 | 16.94 | 30.42 |
| freeze-thawed + bmc's | | | |
| Oral Fibroblasts - | 0.00 | 1.57 | 65.55 |
| MitC treated + bmc's | | | |
| Oral Fibroblasts - | 0.00 | 1.09 | 18.99 |
| MitC treated, grown for 1 week + bmc's | | | |
| Oral Fibroblasts - MitC treated | 0.00 | 0.00 | 0.00 |
| Hypertrophic Chondrocytes - | 0.00 | 0.00 | 0.00 |
| MitC treated | | | |
| Hypertrophic Chondrocytes - | 0.00 | 0.00 | 0.00 |
| freeze-thawed | | | |

TABLE 13
The total surface area of the CFU-Ca and the CFU-f colonies
can be used to determine the % calcification of the
CFU-f surface area for each petri dish.

| | % CFU-f surface area |
|---|---|
| | calcification |
| | % CFU-f calcification |
| Bone Marrow Cells (bmc's) | 1.56 |
| | 2.51 |
| | 2.80 |
| | 3.49 |
| Hypertrophic Chondorcytes - MitC treated + | 71.38 |
| bmc's | |
| | 49.95 |
| | 59.18 |
| Hypertrophic Chondrocytes - MitC treated, | 115.46 |
| grown for 1 week + bmc's | |
| | 64.35 |
| | 82.11 |
| | 91.31 |
| Hypertrophic Chondrocytes - freeze-thawed + | 58.37 |
| bmc's | |
| | 67.80 |
| | 58.39 |
| Hypertrophic Chondrocytes - MitC treated, | 64.06 |
| grown for 1 week freeze-thawed + bmc's | |
| | 70.43 |
| | 74.92 |
| | 67.28 |
| Oral Fibroblasts - MitC treated + bmc's | 0.00 |
| | 0.00 |
| | 0.00 |
| Oral Fibroblats - MitC treated, grown for | 0.00 |
| 1 week + bmc's | |
| | 0.00 |
| | 0.00 |
| | 0.00 |

TABLE 14
The mean % calcification of the CFU-f colony surface area,
for each treatment and control group.

| | The main % CFU-f |
|---|---|
| | colony surface area |
| | % CFU-f calcification |
| Bone Marrow Cells (bmc's) | 2.59 |
| Hypertrophic Chondrocytes - | 60.17 |
| MitC treated + bmc's | |
| Hypertrophic Chondrocytes - | 88.31 |
| MitC treated, grown for 1 week + bmc's | |
| Hypertrophic Chondrocytes - | 64.85 |
| freeze-thawed + bmc's | |
| Hypertrophic Chondrocytes - MitC | 69.17 |
| treated, grown for 1 week freeze-thawed + bmc's | |
| Oral Fibroblasts - MitC treated + bmc's | 0.00 |
| Oral Fibroblasts - | 0.00 |
| MitC treated, grown for 1 week + bmc's | |

TABLE 15
Standard deviation of the results for %
calcification of the CFU-f colony surface area
per treatment and control group.

| | % calcification of CFU-f |
|---|---|
| | colony surface area |
| | Standard Deviation |
| | % CFU-f calcification |
| Bone Marrow Cells (bmc's) | 0.80 |
| Hypertrophic Chondrocytes - | 10.75 |
| MitC treated + bmc's | |
| Hypertrophic Chondrocytes - | 21.28 |
| MitC treated, grown for 1 week + bmc's | |
| Hypertrophic Chondrocytes - | 5.62 |
| freeze-thawed + bmc's | |
| Hypertrophic Chondrocytes - | 4.63 |
| MitC treated, grown for 1 week | |
| freeze-thawed + bmc's | |
| Oral Fibroblasts - | 0.00 |
| MitC treated + bmc's | |
| Oral Fibroblasts - | 0.00 |
| MitC treated, grown for 1 week + bmc's | |

TABLE 16
(SEM) The standard error of the mean %
calcification of the CFU-f colony surface area,
for each treatment and control group.

| | % calcification of CFU-f |
|---|---|
| | colony surface area |
| | Standard error |
| | of the mean |
| | % CFU-f calcification |
| Bone Marrow Cells (bmc's) | 0.4 |
| Hypertrophic Chondrocytes - | 6.2 |
| MitC treated + bmc's | |
| Hypertrophic Chondrocytes - | 10.6 |
| MitC treated, grown for 1 week + bmc's | |
| Hypertrophic Chondrocytes - | 3.2 |
| freeze-thawed + bmc's | |
| Hypertrophic Chondrocytes - | 2.3 |
| MitC treated, grown for | |
| 1 week freeze-thawed + bmc's | |
| Oral Fibroblasts - MitC treated + bmc's | 0 |
| Oral Fibroblasts - | 0 |
| MitC treated, grown for 1 week + bmc's | |

TABLE 17
Percentage CFU-f differentiation

| | differentiation per petri dish | | |
|---|---|---|---|
| | Percentage CFU-f | | |
| | CFU-Ca | CFU-Col | CFU-f |
| Bone Marrow Cells (bmc's) | 52% | 3% | 1% |
| | 71% | 3% | 3% |
| | 57% | 4% | 2% |
| | 55% | 3% | 1% |
| Hypertrophic Chondrocytes - | 104% | 59% | 73% |
| MitC treated + bmc's | | | |
| | 88% | 43% | 68% |
| | 95% | 55% | 77% |
| Hypertrophic Chondrocytes - | 95% | 87% | 100% |
| MitC treated, groen | | | |
| for 1 week + bmc's | | | |
| | 92% | 65% | 103% |
| | 86% | 72% | 93% |
| | 85% | 86% | 88% |
| Hypertrophic Chondrocytes - | 96% | 53% | 50% |
| freeze-thawed + bmc's | | | |
| | 96% | 55% | 56% |
| | 99% | 62% | 45% |
| Hypertrophic Chondrocytes - | 89% | 53% | 71% |
| MitC treated, grown | | | |
| for 1 week freeze-thawed + bmc's | | | |
| | 81% | 65% | 88% |
| | 88% | 66% | 81% |
| | 81% | 59% | 75% |

TABLE 18
Standard deviation of the total colony surface area,
in the sets of 3-4 petri dishes for
each treatment and control group.

| | Percentage CFU-f | | |
|---|---|---|---|
| | differentiation | | |
| | Standard Deviation | | |
| | CFU-Ca | CFU-Col | CFU-f |
| Bone Marrow Cells (bmc's) | 0.09 | 0.01 | 0.01 |
| Hypertrophic Chondrocytes - | 0.05 | 0.11 | 0.07 |
| MitC treated + bmc's | | | |
| Hypertrophic Chondrocytes - | 0.02 | 0.05 | 0.05 |
| MitC treated, grown for 1 week + bmc's | | | |
| Hypertrophic Chondrocytes - | 0.02 | 0.05 | 0.05 |
| freeze-thawed + bmc's | | | |
| Hypertrophic Chondrocytes - | 0.04 | 0.06 | 0.07 |
| MitC treated, grown for 1 week freeze- | | | |
| thawed + bmc's | | | |

TABLE 19
Mean percentage CFU-f differentiation

| | Mean percentage | | |
|---|---|---|---|
| | CFU-f differentiation | | |
| | CFU-Ca | CFU-Col | CFU-f |
| Bone Marrow Cells (bmc's) | 59% | 3% | 2% |
| Hypertrophic Chondrocytes - | 96% | 52% | 73% |
| MitC treated + bmc's | | | |
| Hypertrophic Chondrocytes - | 89% | 78% | 96% |
| freeze-thawed + bmc's | | | |
| Hypertrophic Chondrocytes - | 97% | 57% | 50% |
| MitC treated, gorwn for 1 week + bmc's | | | |
| Hypertrophic Chondorcytes - | 85% | 61% | 79% |
| MitC treated, grown for 1 week | | | |
| freeze-thawed + bmc's | | | |

TABLE 20

| Std/ | | | A450 − | | | | | | | | | Conc |
|---|---|---|---|---|---|---|---|---|---|---|---|---|
| smpl | A-450 | A-540 | A540 | Average | Corrected | Log | Log Conc | Conc | Conc | Prism Log | ConcPzm | (pM) |
| 0 | 0.1282 | 0.1155 | 0.0127 | 0.0174 | | | Y = | −2.5713 | Y = | ÷0.0193 | | |
| | | | | | | | 0.8714x | | 0.0012x | | | |
| 0 | 0.1252 | 0.1031 | 0.0221 | | | | | | | | | |
| 15.6 | 0.1453 | 0.0923 | 0.053 | 0.0494 | 0.032 | −1.49485 | 1.235311 | 17.19139 | 10.58333 | | | |
| 15.6 | 0.1389 | 0.0931 | 0.0458 | | | | | | | | | |
| 31.2 | 0.1558 | 0.0919 | 0.0719 | 0.0545 | −1.263603 | 1.500685 | 31.67266 | 29.33333 | | | | |
| 31.2 | 0.1612 | 0.0813 | 0.0799 | | | | | | | | | |
| 62.5 | 0.2138 | 0.1133 | 0.1005 | 0.11095 | 0.09355 | −1.028956 | 1.769961 | 58.87904 | 61.875 | | | |
| 62.5 | 0.2195 | 0.981 | 0.1214 | | | | | | | | | |
| 125 | 0.2517 | 0.0932 | 0.1685 | 0.17985 | 0.16245 | −0.78928 | 2.045008 | 110.9194 | 119.2917 | | | |
| 125 | 0.2975 | 0.1063 | 0.1912 | | | | | | | | | |
| 250 | 0.4299 | 0.0929 | 0.337 | 0.3341 | 0.3167 | −0.499352 | 2.377723 | 238.629 | 247.8333 | | | |
| 250 | 0.4363 | 0.1051 | 0.3312 | | | | | | | | | |
| 500 | 0.7387 | 0.098 | 0.6407 | 0.6366 | 0.6192 | −0.208169 | 2.711879 | 515.0846 | 499.9167 | | | |
| 500 | 0.7258 | 0.0933 | 0.6325 | | | | | | | | | |
| 1000 | 1.3172 | 0.1087 | 1.2085 | 1.20385 | 1.18645 | 0.074249 | 3.035976 | 1086.365 | 972.625 | | | |
| 1000 | 1.305 | 0.1058 | 1.1992 | | | | | | | | | |
| LQC | 0.2769 | 0.1153 | 0.1616 | 0.1871 | 0.1697 | −0.770318 | 2.066768 | 116.6187 | 125.3333 | −9.897568 | 1.266E−10 | 126.5995 |
| H20 | | | | | | | | | | | | |
| LQC | 0.3307 | 0.1181 | 0.2126 | | | | | | | | | |
| H20 | | | | | | | | | | | | |
| MQC | 0.6697 | 0.1227 | 0.547 | 0.5275 | 0.5101 | −0.292345 | 2.61528 | 412.3637 | 409 | −9.380689 | 4.1621E−10 | 416.2086 |
| H20 | | | | | | | | | | | | |
| MQC | 0.6178 | 0.1098 | 0.508 | | | | | | | | | |
| H20 | | | | | | | | | | | | |
| HQC | 0.9334 | 0.1178 | 0.8156 | 0.83575 | 0.81835 | −0.87061 | 2.85086 | 709.3485 | 665.875 | −9.168353 | 6.7865E−10 | 678.6518 |
| H20 | | | | | | | | | | | | |
| HQC | 0.9559 | 0.1 | 0.8559 | | | | | | | | | |
| H20 | | | | | | | | | | | | |
| LQC | 0.2691 | 0.1011 | 0.168 | 0.17385 | 0.15645 | −0.805624 | 2.026252 | 106.2311 | 114.2917 | −9.937621 | 1.1545E−10 | 115.446 |
| T5% | | | | | | | | | | | | |
| LQC | 0.2789 | 0.0992 | 0.1797 | | | | | | | | | |
| T5% | | | | | | | | | | | | |
| MQC | 0.4642 | 0.1077 | 0.3565 | 0.35455 | 0.33715 | −0.472177 | 2.408909 | 256.3946 | 264.875 | −9.570548 | 2.8881E−10 | 268.8141 |
| T5% | | | | | | | | | | | | |
| MQC | 0.465 | 0.1124 | 0.3525 | | | | | | | | | |
| T5% | | | | | | | | | | | | |
| HQC | 0.7847 | 0.1184 | 0.6663 | 0.86095 | 0.64355 | −0.191418 | 2.731102 | 538.3962 | 520.2083 | −9.275826 | 5.2988E−10 | 529.8757 |
| T5% | | | | | | | | | | | | |
| MQC | 0.7607 | 0.1051 | 0.6556 | | | | | | | | | |
| T5% | | | | | | | | | | | | |
| 409 g/ | 0.108 | 0.0963 | 0.0117 | 0.014 | −0.0034 | #Num | #Num | #Num | −18.91667 | | | |
| CPs | | | | | | | | | | | | |
| 40 pg/ | 0.1005 | 0.0842 | 0.0163 | | | | | | | | | |
| CPs | | | | | | | | | | | | |
| 80 pg/ | 0.1231 | 0.0957 | 0.0274 | 0.02325 | 0.00585 | −2.232844 | 0.388405 | 2.445709 | −11.20833 | | | |
| CPs | | | | | | | | | | | | |
| 80 pg/ | 0.1118 | 0.0927 | 0.0191 | | | | | | | | | |
| CPs | | | | | | | | | | | | |
| 120 pg/ | 0.1123 | 0.0963 | 0.016 | 0.01115 | −0.00625 | #Num | #Num | #Num | −21.29167 | | | |
| CPS | | | | | | | | | | | | |
| 120 pg/ | 0.1124 | 0.1061 | 0.0063 | | | | | | | | | |
| CPs | | | | | | | | | | | | |
| 160 pg/ | 0.1223 | 0.0971 | 0.0252 | 0.02665 | 0.00925 | −2.033858 | 0.616757 | 4.137677 | −8.375 | | | |
| CPs | | | | | | | | | | | | |
| 160 pg/ | 0.1104 | 0.0942 | 0.0281 | | | | | | | | | |
| CPs | | | | | | | | | | | | |
| 40 pg/ | 0.1632 | 0.0921 | 0.0183 | 0.0453 | 0.0279 | −1.554396 | 1.166978 | 14.6885 | 7.166667 | | | |
| T5% | | | | | | | | | | | | |
| 40 pg/ | 0.1757 | 0.0909 | 0.0723 | | | | | | | | | |
| T5% | | | | | | | | | | | | |
| 80 pg/ | 0.2257 | 0.0818 | 0.0939 | 0.1195 | 0.1021 | −0.990974 | 1.813548 | 65.09506 | 69 | −10.15378 | 7.0181E−11 | 70.18107 |
| T5% | | | | | | | | | | | | |
| 80 pg/ | 0.2059 | 0.0806 | 0.1451 | | | | | | | | | |
| T5% | | | | | | | | | | | | |
| 120 pg/ | 0.2336 | 0.0857 | 0.1202 | 0.1288 | 0.1114 | −0.953115 | 1.856995 | 71.94402 | 76.75 | −10.10875 | 7.7848E−11 | 77.84846 |
| T5% | | | | | | | | | | | | |
| 120 pg/ | 0.2482 | 0.0962 | 0.1374 | | | | | | | | | |
| T5% | | | | | | | | | | | | |
| 160 pg/ | 0.3067 | 0.0958 | 0.1524 | 0.18825 | 0.17085 | −0.767385 | 2.070134 | 117.5251 | 126.2917 | −9.894258 | 1.2757E−10 | 127.5681 |
| T5% | | | | | | | | | | | | |
| 160 pg/ | 0.3009 | 0.0825 | 0.2241 | | | | | | | | | |
| T5% | | | | | | | | | | | | |
| 40 pg/ | 0.1129 | 0.0817 | 0.2192 | 0.12555 | 0.10815 | −0.965973 | 1.842238 | 69.54059 | 74.04167 | −10.12399 | 7.5164E−11 | 75.16402 |
| T&C | | | | | | | | | | | | |
| 40 pg/ | 0.1148 | 0.081 | 0.0319 | | | | | | | | | |
| T&C | | | | | | | | | | | | |
| 80 pg/ | 0.1183 | 0.009 | 0.1058 | 0.0638 | 0.0464 | −1.333482 | 1.420493 | 26.33258 | 22.58333 | −10.56767 | 2.5842E−11 | 25.84223 |
| T&C | | | | | | | | | | | | |
| 80 pg/ | 0.1517 | 0.0965 | 0.0218 | | | | | | | | | |
| T&C | | | | | | | | | | | | |
| 120 pg/ | 0.1052 | 0.0789 | 0.0728 | 0.04695 | 0.02955 | −1.529443 | 1.195613 | 15.68965 | 8.541667 | | | |
| T&C | | | | | | | | | | | | |
| 120 pg/ | 0.119 | 0.0841 | 0.0211 | | | | | | | | | |
| T&C | | | | | | | | | | | | |
| 160 pg/ | 0.1596 | 0.0964 | 0.0226 | 0.0523 | 0.0349 | −1.457175 | 1.278547 | 18.99094 | 13 | −10.75932 | 1.7405E−11 | 17.40524 |
| T&C | | | | | | | | | | | | |
| 160 pg/ | 0.1326 | 0.0776 | 0.082 | | | | | | | | | |
| T&C | | | | | | | | | | | | |
| Neat | 0.3599 | 0.0896 | 0.043 | 0.15995 | 0.14255 | −0.846033 | 1.97988 | 95.47282 | 102.7083 | 9.983869 | 1.0378E−10 | 103.7841 |
| M wet | | | | | | | | | | | | |
| Neat | 0.3281 | 0.083 | 0.2769 | | | | | | | | | |
| M wet | | | | | | | | | | | | |
| HQC | 0.3352 | 0.0913 | 0.2368 | 0.24045 | 0.22305 | −0.651598 | 2.203009 | 159.5913 | 169.7917 | −9.765138 | 1.7174E−10 | 171.7363 |
| cps | | | | | | | | | | | | |
| HQC | 0.2997 | 0.0911 | 0.2441 | | | | | | | | | |
| cps | | | | | | | | | | | | |

1. An extracellular material obtained from skeletal cells, which material has osseoinductive bone repair/regeneration activity in vivo, and is in freeze-dried (lyophilyzed) form.

2. A material according to claim 1 which is obtained from cartilage cells.

3. A material according to claim 1 which is obtained from hypertrophic cartilage cells.

4. A material according to claim 1 which is obtained from immortalised hypertrophic chondrocyte cells.

5. A material according to claim 1 which is obtained from a human cell.

6. A material according to claim 1 which is obtained from a human cell line.

7. A material according to claim 1 which contains a mixture of: (1) one or more cytokine; (2) one or more growth factor; and (3) one or more collagen.

8. A therapeutic composition which comprises an active ingredient which is an effective amount of an osseoinductive material according to claim 1.

9. A therapeutic composition according to claim 8 which includes a physiologically acceptable excipient and/or adjuvant and/or carrier.

10. A composition according to claim 8 in frozen form.

11. A composition according to claim 9 in frozen form.

12. A composition according to claim 8 in frozen-thawed form.

13. A composition according to claim 9 in frozen-thawed form.

14. A composition according to claim 8 in freeze-dried form.

15. A composition according to claim 9 in freeze-dried form.

16. A method for producing osseoinductive extracellular material from skeletal cells which method comprises the steps of:
(1) culturing skeletal cells in a suitable culture medium;
(2) harvesting extracellular material produced by said cultured cells; and optionally isolating and/or purifying said harvested material; and
(3) lyophilizing said material.

17. A method according to claim 16 including the additional step of:
(4) adding a physiologically acceptable excipient and/or adjuvant and/or carrier, to form a therapeutic composition.

18. A material produced by the method of claim 16.

19. A composition produced by the method of claim 17.

20. A method of treating a patient (human or other animal) requiring bone repair/regeneration, which involves administering to said patient an osseoinductive amount of a material according to claim 1.

21. A method of treating a patient (human or other animal) requiring bone repair/regeneration, which involves administering to said patient an osseoinductive amount of a composition according to claim 8.

22. (canceled).
